SEQUENCE LISTING (1 sequence)

SEQ ID NO: 1 - DNA, 27 bp, Artificial Sequence
  Transcription Factor Double-stranded DNA Binding Template
ccgatttagcaaaacgtcgcatcggtc

(19) United States
(12) Patent Application Publication
(10) Pub. No.: US 2011/0059962 A1
(43) Pub. Date: Mar. 10, 2011

(54) TRANSCRIPTION FACTOR MODULATING COMPOUNDS AND METHODS OF USE THEREOF

(76) Inventors: MICHAEL N. ALEKSHUN, MARLBORO, NJ (US); VICTORIA BARTLETT, FRANKLIN, MA (US); MICHAEL P. DRAPER, PLAISTOW, NH (US); LYNNE GARRITY-RYAN, MELROSE, MA (US); RAINA GAY, CHARLESTOWN, MA (US); MARK GRIER, MEDFORD, MA (US); OAK K. KIM, CAMBRIDGE, MA (US); STUART B. LEVY, BOSTON, MA (US)

(21) Appl. No.: 12/765,528

(22) Filed: Apr. 22, 2010

Related U.S. Application Data
(60) Provisional application No. 61/171,825, filed on Apr. 22, 2009.

(51) Int. Cl. C07D 235/22; A61K 31/4184; A61K 31/4439; C07D 401/12; A61K 31/5377; C07D 413/12; A61K 31/4545; C07D 403/12; A61K 31/496; C12N 1/00; C12N 1/20; A61P 31/04

(52) U.S. Cl. 514/234.5; 548/309.7; 514/395; 514/338; 546/273.4; 544/131; 514/318; 546/194; 544/364; 514/253.09; 435/243; 435/252.1

(57) ABSTRACT

Substituted benzoimidazole compounds useful as anti-infectives that decrease resistance, virulence, or growth of microbes are provided. Methods of using substituted benzimidazole compounds, in, e.g., reducing virulence and infectivity, inhibiting biofilms and treating bacterial infections, are also provided.

### RELATED APPLICATIONS

This application claims priority to U.S. Patent Provisional Application No. 61/171,825, filed on Apr. 22, 2009. The contents of the foregoing application are hereby incorporated in their entirety.

### BACKGROUND OF THE INVENTION

Most antibiotics currently used and in development to treat bacterial infections impose selective pressure on microorganisms and have led to the development of widespread antibiotic resistance. Therefore, the development of an alternative approach to treating microbial infections would be of great benefit.

Multidrug resistance in bacteria is generally attributed to the acquisition of multiple transposons and plasmids bearing genetic determinants for different mechanisms of resistance (Gold et al. 1996. N. Engl. J. Med. 335:1445). However, descriptions of intrinsic mechanisms that confer multidrug resistance have begun to emerge. The first of these was a chromosomally encoded multiple antibiotic resistance (mar) locus in Escherichia coli (George and Levy, 1983. J. Bacteriol. 155:531; George and Levy, 1983 J. Bacteriol. 155:541). Mar mutants of E. coli arose at a frequency of 10⁻⁶ to 10⁻⁷ and were selected by growth on subinhibitory levels of tetracycline or chloramphenicol (George and Levy, supra). These mutants exhibited resistance to tetracyclines, chloramphenicol, penicillins, cephalosporins, puromycin, nalidixic acid, and rifampin (George and Levy, supra). Later, the resistance phenotype was extended to include fluoroquinolones (Cohen et al. 1989. Antimicrob. Agents Chemother. 33:1318), oxidative stress agents (Ariza et al. 1994. J. Bacteriol. 176:143; Greenberg et al. 1991. J. Bacteriol. 73:4433), and more recently, organic solvents (White et al. 1997. J. of Bacteriology 179:6122; Asako, et al. 1997. J. Bacteriol. 176:143) and household disinfectants, e.g., pine oil and/or TRICLOSAN® (McMurry et al. 1998. FEMS Microbiology Letters 166:305; Moken et al. 1997. Antimicrobial Agents and Chemotherapy 41:2770).

The mar locus consists of two divergently positioned transcriptional units that flank a common promoter/operator region in E. coli, Salmonella typhimurium, and other Entrobacteriacae (Alekshun and Levy. 1997, Antimicrobial Agents and Chemother. 41: 2067). One operon encodes MarC, a putative integral inner membrane protein without any yet apparent function, but which appears to contribute to the Mar phenotype in some strains. The other operon comprises marRAB, encoding the Mar repressor (MarR), which binds marO and negatively regulates expression of marRAB (Cohen et al. 1994. J. Bacteriol. 175:1484; Martin and Rosner, 1995. PNAS 92:5456; Seoane and Levy, 1995 J. Bacteriol. 177:530), an activator (MarA), which controls expression of other genes on the chromosome, e.g., the mar regulon (Cohen et al. 1994 J. Bacteriol. 175:1484; Gambino et al. 1993. J. Bacteriol. 175:2888; Seoane and Levy, 1995 J. Bacteriol. 177:530), and a putative small protein (MarB) of unknown function.

Exposure of E. coli to several chemicals, including tetracycline and chloramphenicol (Hachler et al. 1991. J. Bacteriol. 173(17):5532-8; Ariza, 1994. J. Bacteriol. 176(1):143-8), sodium salicylate and its derivatives (Cohen, 1993. J. Bacteriol. 175(24):7856-62) and oxidative stress agents (Seoane et al. 1995. J Bacteriol; 177(12):3414-9) induces the Mar phenotype. Some of these chemicals act directly at the level of MarR by interacting with the repressor and inactivating its function (Alekshun, 1999. J. Bacteriol. 181:3303-3306) while others (antibiotics such as tetracycline and chloramphenicol) appear to induce mar expression by an alternative mechanism (Alekshun, 1999. J. Bacteriol. 181:3303-3306) e.g., through a signal transduction pathway.

Once expressed, MarA activates the transcription of several genes that constitute the E. coli mar regulon (Alekshun, 1997. Antimicrob. Agents Chemother. 41:2067-2075; Alekshun, 1999. J. Bacteriol. 181:3303-3306). With respect to decreased antibiotic susceptibility, the increased expression of the AcrAB/TolC multidrug efflux system (Fralick, 1996. J. Bacteriol. 178(19):5803-5; Okusu, 1996. J. Bacteriol. 178(1):306-8) and decreased synthesis of OmpF (Cohen, 1988. J. Bacteriol. 170(12):5416-22) an outer membrane protein, play major roles. Organic solvent tolerance, however, is attributed to MarA mediating increased expression of AcrAB, ToIC, OmpX, and a 77 kDa protein (Aono, 1998. Extremophiles 2(3):239-48; Aono, 1998. J. Bacteriol. 180(4):938-44) but is independent of OmpF levels (Asako, 1999. Appl. Environ. Microbiol. 65(1):294-6).

MarA is a member of the AraC/XylS family of transcriptional activators (Gallegos et al. 1993. Nucleic Acids Res. 21:807). There are more than 100 proteins within the AraC/XylS family and a defining characteristic of this group of proteins is the presence of two helix-turn-helix (HTH) DNA binding motifs. Proteins within this family activate many different genes, some of which produce antibiotic and oxidative stress resistance or control microbial metabolism and virulence (Gallegos et al. supra). MarA (AraC) family proteins are present in nearly all clinically important bacteria including Pseudomonas aeruginosa, Yersinia spp., E. coli (including enteroaggregative, enterotoxigenic, and enteropathogenic strains), Klebsiella spp., Shigella spp., Salmonella spp., Vibrio cholerae, Staphylococcus aureus, and Streptococcus pneumoniae (M.-T. Gallegos et al. 1993. Nuc. Acids. Res. 21:807). Inactivation of MarA (AraC) family proteins by mutation attenuates virulence of bacteria in various animal models of infection (P. Casaz et al. 2006. Microbiol. 152:3643; G. A. Champion et al. 2003. Mol. Micro. 23:323; Y. Flashner et al. 2004. Infect. Immun. 72:908; D. S. Bieber et al. 1998. Sci. 280:2114).

MarA, Rob, and SoxS proteins are required for full E. coli virulence in a murine ascending pyelonephritis model (P. Casaz et al. 2006. Microbiol. 152:3643). Deletion of genes for marA, rob, and soxS from a clinical (intestinal fistula) E. coli isolate (KM-D), removed its ability to colonize the kidneys. Wild type virulence was restored when the deletion strain (SRM) was complemented with a single chromosomal copy of the marA, soxS, or rob genes.

The Y. pseudotuberculosis MarA (AraC) family protein LcrF (also called VirF in Y. enterocolitica) regulates expression of a major virulence determinant, the type III secretion system (TTSS) (G. R. Cornelis and H. Wolf-Watz, 1997. Mol. Microbiol. 23:861-867). The TTSS delivers toxins directly into host cells via a needle-like apparatus. Mutants that do not express the TTSS show dramatic attenuation of virulence in whole cell and animal models of infection (G. R. Cornelis and H. Wolf-Watz, 1997. Mol. Microbiol. 23:861-867; L. K. Logsdon and J. Mecsas, 2003. Infect. Immun. 71:4595-4607; J. Mecsas et al. 2001. Infect. Immun., 69:2779-2787; D. M. Monack et al. 1997. Proc. Natl. Acad. Sci. U.S.A. 94:10385-10390). Flashner et al. have recently investigated the effects lcrF deletion on the pathogenicity of Y. pestis in a mouse model of septic infection (Y. Flashner et al. 2004. Infect. Immun. 72:908-915). The LD₅₀ (50% lethal dose) of wild type Y. pestis in this model is approximately 1 colony forming unit (CFU). When an 1:1 mixture of wild type and lcrF mutant Y. pestis was used to infect mice, the competitive index (defined as the ratio of lcrF/wt recovered following infection vs. the ratio of lcrF/wt used for infection) was <10⁻⁷ indicating severe attenuation of the mutant organism.

The Pseudomonas aeruginosa MarA (AraC) family protein ExsA regulates expression of a well established virulence determinant, the type III secretion system (T. L. Yahr et al. 2006. Mol. Micro. 62(3):631). Mutants lacking ExsA show dramatically reduced virulence in animal models of P. aeruginosa infection (V. J. Finck-Barbancon et al. 1997. Mol. Micro. 25(3):547; A. R. Hauser et al. 1998. Mol. Micro. 27:807; I. Kudoh et al. 1994. Am. J. Physiol. 267:L551; M. A. Laskowski et al. 2004. Mol. Micro. 54(4):1090; E. J. Lee et al. 2003. Invest. Ophthalmol. Vis. Sci. 44(9):3892; R. S. Smith et al. 2004. Infect. & Immun. 72(3):1677). Furthermore, expression of the type III secretion system is correlated with increased severity of disease in clinical pneumonia cases, including ventilator-associated pneumonia (A. R. Hauser et al. 2002. Crit. Care Med. 30(3):521; G. S. Schulert et al. 2003. J. Infect. Dis. 188:1695; A. Roy-Burman et al. 2001. J. Infect. Dis. 183:1767).

### SUMMARY OF THE INVENTION

The present invention pertains, at least in part, to transcription factor modulating compounds and pharmaceutical compositions thereof.

The present invention also pertains, at least in part, to a method for reducing infectivity and/or virulence of a microbial cell by contacting the cell with a transcription factor modulating compound.

In another embodiment, the present invention pertains, at least in part, to a method for modulating transcription of genes regulated by one or more transcription factors in the MarA (AraC) family. The method includes contacting a transcription factor with a transcription factor modulating compound. Specifically, in one embodiment, the transcription factor is ExsA, LcrF (VirF) or SoxS.

The present invention also pertains, at least in part, to a method for preventing bacterial growth on a contact lens by administering a composition comprising an acceptable carrier and a transcription factor modulating compound.

The present invention also pertains, at least in part, to a method for preventing or treating an infection in a patient into which an indwelling device has been implanted (e.g., ventilator-associated pneumonia in patients receiving mechanical ventilation) by administering a composition comprising a transcription factor modulating compound. The present invention also pertains, at least in part, to methods for treating or preventing biofilm formation in a subject by administering to the subject an effective amount of a transcription factor modulating compound.

In another embodiment, the present invention pertains, at least in part, to a method for treating or preventing a bacterial infection in a subject by administering to the subject an effective amount of a transcription factor modulating compound.

The present invention also pertains, at least in part, to a method for prevention or treatment of a urinary tract infection in a subject by administering to the subject an effective amount of a transcription factor compound.

In yet another embodiment, the invention pertains, at least in part, to a method for treating or preventing pneumonia in a subject by administering to the subject an effective amount of a transcription factor modulating compound.

In a further embodiment, the invention pertains, at least in part, to a method for treating burn wounds and corneal ulcers in a subject by administering to the subject an effective amount of a transcription factor modulating compound.

In another embodiment, the present invention pertains, at least at part, to a method for treating or preventing ascending pyelonephritis or kidney infection in a subject by administering to the subject an effective amount of a transcription factor modulating compound.

In one embodiment, the present invention pertains, at least in part, to a method for inhibiting a MarA family polypeptide by contacting a Mar family polypeptide with an effective amount of a transcription factor modulating compound.

The invention also pertains, at least in part, to a pharmaceutical composition comprising a pharmaceutically acceptable carrier and a transcription factor modulating compound.

### DETAILED DESCRIPTION OF THE INVENTION

The Mar proteins are members of the AraC family of bacterial transcription regulators characterized by two highly conserved helix-turn-helix (HTH) DNA-binding domains. The signaling networks regulating the activity of Mar proteins vary and, while there is high conservation within the DNA binding domains, all Mar proteins bind to distinct DNA sequences in the promoter regions of the genes which they regulate. Mar proteins are present in all clinically important bacteria whose genomes have been examined including Pseudomonas aeruginosa, Yersinia spp., E. coli (including enteroaggregative, enterotoxigenic and enteropathogenic strains), Klebsiella spp., Shigella spp., Salmonella spp., Vibrio cholerae, Staphylococcus aureus and Streptococcus pneumoniae. Mar proteins confer upon bacteria the ability to cause infections, resist antibiotics and adapt to hostile environments. Inactivation of Mar proteins by mutation attenuates the virulence of bacterial pathogens in animal models of infection, but does not affect bacterial growth.

The invention relates to anti-infective transcription factor modulating compounds that target the virulence and infectivity of a microbial cell, thus preventing infection or disease in a subject. The invention pertains, at least in part, to a method for reducing the infectivity or virulence of a microbial cell, comprising contacting said cell with a transcription factor modulating compound, e.g. a compound of formula I, II, III, IV, IVa, VI, VIa, VII, or VIII or a compound of Table 2. The term “reducing infectivity” includes decreasing or eliminating the potential of a microbial cell to cause an infection. The term “reducing virulence” includes decreasing or eliminating the ability of a microbial cell to cause disease. Examples of microbial cells, include, but are not limited to, E. coli, Y. pseudotuberculosis, Y. pestis, Klebsiella pneumoniae, Acinetobacter baumannii and P. aeruginosa. A skilled artisan, using routine techniques, would be able to determine whether a microbial cell is infective or virulent.

In one embodiment, the method of reducing infectivity or virulence of a microbial cell includes reducing the manner in which a microbial cell causes a disease. Without being bound by theory, the methods for reducing infectivity or virulence of a microbial cell may include, for example, the inhibition of the adhesion of a microbial cell to a host cell; the inhibition of the colonization of the microbial cell in the host; the inhibition of the microbial cell from entering host cells and/or entry into the host body; the reduction or elimination of the ability of the microbial cell to produce immune response inhibitors or toxins that may cause tissue damage or damage to the host cells. The term “microbe” includes microorganisms that cause disease. For example, in one embodiment, microbes are unicellular and include bacteria, fungi, or protozoa. In another embodiment, microbes suitable for use in the invention are multicellular, e.g., parasites or fungi. In another embodiment, microbes are pathogenic for humans, animals, or plants. In one embodiment, the microbes include prokaryotic organisms. In other embodiments, the microbes include eukaryotic organisms. In a further embodiment, the microbe is antibiotic resistant.

In one embodiment, microbes against which a transcription factor modulating compound of the invention may be used are bacteria, e.g., Gram negative or Gram positive bacteria. In one embodiment, the microbe includes any bacteria that are shown to become resistant to antibiotics, e.g., display a Mar phenotype or are infectious or potentially infectious. Exemplary bacteria that contain MarA homologs include the following: E. coli (e.g., UPEC (uropathogenic) or EPEC (enteropathogenic)), Salmonella enterica (e.g., Cholerasuis(septicemia), Enteritidis enteritis, Typhimurium enteritis, Typhimurium (multi-drug resistant)), Yersinia enterocolitica, Yersinia pestis, Yersinia pseudotuberculosis, Pseudomonas aeruginosa, Enterobacter spp., Klebsiella sp., Proteus spp., Vibrio cholerae, Shigella sp., Providencia stuartii, Neisseria meningitides, Mycobacterium tuberculosis, Mycobacterium leprae, Staphylococcus aureus, Streptococcus pyogenes, Enterococcus faecalis, Bordetella pertussis and Bordetella bronchiseptica.

Examples of microbes against which a transcription factor modulating compound of the invention may be used include, but are not limited to, Pseudomonas aeruginosa, Pseudomonas fluorescens, Pseudomonas acidovorans, Pseudomonas alcaligenes, Pseudomonas putida, Stenotrophomonas maltophilia, Burkholderia cepacia, Aeromonas hydrophilia, Escherichia coli, Citrobacter freundii, Salmonella typhimurium, Salmonella typhi, Salmonella paratyphi, Salmonella enteritidis, Shigella dysenteriae, Shigella flexneri, Shigella sonnei, Enterobacter cloacae, Enterobacter aerogenes, Klebsiella pneumoniae, Klebsiella oxytoca, Serratia marcescens, Francisella tularensis, Morganella morganii, Proteus mirabilis, Proteus vulgaris, Providencia alcalifaciens, Providencia rettgeri, Providencia stuartii, Acinetobacter calcoaceticus, Acinetobacter baumannii, Acinetobacter haemolyticus, Yersinia enterocolitica, Yersinia pestis, Yersinia pseudotuberculosis, Yersinia intermedia, Bordetella pertussis, Bordetella parapertussis, Bordetella bronchiseptica, Haemophilus influenzae, Haemophilus par ainfluenzae, Haemophilus haemolyticus, Haemophilus parahaemolyticus, Haemophilus ducreyi, Pasteurella multocida, Pasteurella haemolytica, Branhamella catarrhalis, Helicobacter pylori, Campylobacter fetus, Campylobacter jejuni, Campylobacter coli, Borrelia burgdorferi, Vibrio cholerae, Vibrio parahaemolyticus, Legionella pneumophila, Listeria monocytogenes, Neisseria gonorrhoeae, Neisseria meningitidis, Gardnerella vaginalis, Bacteroides fragilis, Bacteroides distasonis, Bacteroides 3452A homology group, Bacteroides vulgatus, Bacteroides ovalus, Bacteroides thetaiotaomicron, Bacteroides uniformis, Bacteroides eggerthii, Bacteroides splanchnicus, Clostridium difficile, Mycobacterium tuberculosis, Mycobacterium avium, Mycobacterium intracellular, Mycobacterium leprae, Corynebacterium diphtheriae, Corynebacterium ulcerans, Streptococcus pneumoniae, Streptococcus agalactiae, Streptococcus pyogenes, Enterococcus faecalis, Enterococcus faecium, Staphylococcus aureus, Staphylococcus epidermidis, Staphylococcus saprophyticus, Staphylococcus intermedius, Staphylococcus hyicus subsp. hyicus, Staphylococcus haemolyticus, Staphylococcus hominis, and Staphylococcus saccharolyticus.

In one embodiment, microbes against which a transcription factor modulating compound of the invention may be used are bacteria from the family Enterobacteriaceae. In preferred embodiments, the compound is effective against a bacteria of a genus selected from the group consisting of Escherichia, Proteus, Salmonella, Klebsiella, Providencia, Enterobacter, Burkholderia, Pseudomonas, Aeromonas, Haemophilus, Yersinia, Acinetobacter, Neisseria and Mycobacteria.

In yet other embodiments, the microbes against which a transcription factor modulating compound of the invention may be used are Gram positive bacteria and are selected from a genus selected from the group consisting of Lactobacillus, Azorhizobium, Streptomyces, Pediococcus, Photobacterium, Haemophilus, Bacillus, Enterococcus, Staphylococcus, Clostridium and Streptococcus.

In other embodiments, the microbes against which a transcription factor modulating compound of the invention may be used are fungi. In one embodiment, the fungus is from the genus Mucor or Candida, e.g., Mucor racmeosus or Candida albicans.

In yet other embodiments, the microbes against which a transcription factor modulating compound of the invention may be used are protozoa. In a preferred embodiment the microbe is a malaria or cryptosporidium parasite.

The term “transcription factor” includes proteins that are involved in gene regulation in both prokaryotic and eukaryotic organisms. Preferably, a transcription factor against which a modulating compound of the invention is effective is present only in a prokaryotic organism. In one embodiment, transcription factors can have a positive effect on gene expression and, thus, may be referred to as an “activator” or a “transcriptional activation factor.” In another embodiment, a transcription factor can negatively affect gene expression and, thus, may be referred to as a “repressor” or a “transcription repression factor.” Activators and repressors are generally used terms and their functions are discerned by those skilled in the art. In one embodiment, the transcription factor is ExsA, SoxS or LcrF (VirF).

Some major families of transcription factors found in bacteria include the helix-turn-helix transcription factors (HTH) (S. C. Harrison and A. K. Aggarwal, 1990. Annual Review of Biochemistry 59:933-969) such as AraC, MarA, Rob, SoxS and LysR; winged helix transcription factors (K. S. Gajiwala and S. K. Burley, 2000. 10:110-116), e.g., MarR, Sar/Rot family, and OmpR (J. L. Huffman and R. G. Brennan 2002. Curr Opin Struct Biol. 12:98-106, E. Martinez-Hackert and A. M. Stock, 1997. Structure. 5:109-124); and looped-hinge helix transcription factors (J. L. Huffman and R. G. Brennan 2002. Curr. Opin. Struct. Biol. 12:98-106), e.g., the AbrB protein family.

MarA (AraC) family proteins are present in nearly all clinically important bacteria including Pseudomonas aeruginosa, Yersinia spp., E. coli (including enteroaggregative, enterotoxigenic, and enteropathogenic strains), Klebsiella spp., Shigella spp., Salmonella spp., Vibrio cholerae, Staphylococcus aureus, and Streptococcus pneumoniae (M.-T. Gallegos et al. 1993. Nuc. Acids. Res. 21:807.). MarA (AraC) family proteins confer upon bacteria the ability to cause infections, resist antibiotics, and adapt to hostile environments. Inactivation of MarA (AraC) family proteins by mutation attenuates virulence of bacteria in various animal models of infection (P. Casaz et al. 2006. Microbiol. 152:3643; G. A. Champion et al. 2003. Mol. Micro. 23:323; Y. Flashner et al. 2004. Infect. Immun. 72:908; D. S. Bieber et al. 1998. Sci. 280:2114).

The terms “AraC family polypeptide,” “AraC/XylS family polypeptide” or “AraC/XylS family peptide” include an art recognized group of prokaryotic transcription factors which contains more than 100 different proteins (Gallegos et al., (1997) Micro. Mol. Biol. Rev. 61: 393; Martin and Rosner, 2001. Curr. Opin. Microbiol. 4:132). AraC family polypeptides include proteins defined in the PROSITE (PS) database as profile PSOI 124. The AraC family polypeptides also include polypeptides described in PS0041, HTH AraC Family 1, PSO 1124 and HTH AraC Family 2.

AraC family proteins contain a conserved DNA binding domain with two helix-turn-helix motifs. This conserved domain spans 100 amino acids with 17 residues showing a high degree of conservation over that span representing the consensus for the family. The overall similarity of the DNA binding domain is >20% among members of the AraC family. For example, ExsA and VirF are 56% identical, 72% similarity across a 266 amino acid overlap and they show 85% identity and 97% similarity in the 100 bp DNA binding domain; VirF and MarA show 23% identity, 42% similarity across a 96 amino acid overlap; and ExsA and MarA show 23% identity, 42% similarity across a 92 amino acid overlap.

In an embodiment, the AraC family polypeptides are generally comprised of, at the level of primary sequence, a conserved stretch of about 100 amino acids, which are believed to be responsible for the DNA binding activity of this protein (Gallegos et al., 1997. Micro. Mol. Biol. Rev. 61:393; Martin and Rosner, 2001. Curr. Opin. Microbiol. 4: 132). AraC family polypeptides also may include two helix-turn-helix DNA binding motifs (Martin and Rosner, 2001. Curr. Opin. Microbiol. 4:132; Gallegos et al. 1997. Micro. Mol. Biol. Rev. 61:393; Kwon et al. 2000. Nat. Struct. Biol. 7:424; Rhee et al. 1998. Proc. Natl. Acad. Sci. U.S.A. 95:10413). The term includes MarA family polypeptides and HTH proteins.

The terms “helix-turn-helix protein,” “HTH protein,” “helix-turn-helix polypeptides” and “HTH polypeptides,” include proteins comprising one or more helix-turn-helix domains. Helix-turn-helix domains are known in the art and have been implicated in DNA binding (Ann. Rev. of Biochem. 1984. 53:293). In one embodiment, a helix-turn-helix domain containing protein is a Mar A family polypeptide. The language “MarA family polypeptide” includes the many naturally occurring HTH proteins, such as transcription regulation proteins which have sequence similarities to MarA and which contain the MarA family signature pattern, which can also be referred to as an AraC/XylS signature pattern. MarA family polypeptides have two “helix-turn-helix” domains. This signature pattern was derived from the region that follows the first, most amino terminal, helix-turn-helix domain (HTH1) and includes the totality of the second, most carboxy terminal helix-turn-helix domain (HTH2). (See PROSITE PS00041).

The MarA family of proteins (“MarA family polypeptides”) represent one subset of AraC/XylS family polypeptides and include proteins like MarA, SoxS, Rob, RamA, AarP, PqrA, etc. The MarA family polypeptides, generally, are involved in regulating resistance to antibiotics, organic solvents, and oxidative stress agents (Alekshun and Levy, 1997. Antimicrob. Agents. Chemother. 41:2067). Like other AraC/XylS family polypeptides, MarA-like proteins also generally contain two HTH motifs as exemplified by the MarA and Rob crystal structures (Kwon et al. 2000. Nat. Struct. Biol. 7:424; Rhee et al. 1998. Proc. Natl. Acad. Sci. U.S.A. 95:10413). Members of the MarA family can be identified by those skilled in the art and will generally be represented by proteins with homology to amino acids 30-76 and 77-106 of MarA. Preferably, a MarA family polypeptide or portion thereof comprises the first MarA family HTH domain (HTH11) (Brunelle, 1989. J. Mol. Biol. 209(4):607-22). In another embodiment, a MarA polypeptide comprises the second MarA family HTH domain (HTH2) (Caswell, 1992. Biochem. J. 287:493-509). In a preferred embodiment, a MarA polypeptide comprises both the first and second MarA family HTH domains.

MarA family polypeptide sequences are “structurally related” to one or more known MarA family members, preferably to MarA. This relatedness can be shown by sequence or structural similarity between two MarA family polypeptide sequences or between two MarA family nucleotide sequences that specify such polypeptides. Sequence similarity can be shown, e.g., by optimally aligning MarA family member sequences using an alignment program for purposes of comparison and comparing corresponding positions. To determine the degree of similarity between sequences, they will be aligned for optimal comparison purposes (e.g., gaps may be introduced in the sequence of one protein for nucleic acid molecule for optimal alignment with the other protein or nucleic acid molecules). The amino acid residues or bases and corresponding amino acid positions or bases are then compared. When a position in one sequence is occupied by the same amino acid residue or by the same base as the corresponding position in the other sequence, then the molecules are identical at that position. If amino acid residues are not identical, they may be similar. As used herein, an amino acid residue is “similar” to another amino acid residue if the two amino acid residues are members of the same family of residues having similar side chains. Families of amino acid residues having similar side chains have been defined in the art (see, for example, Altschul et al. 1990. J. Mol. Biol. 215:403), including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan). The degree (percentage) of similarity between sequences, therefore, is a function of the number of identical or similar positions shared by two sequences (i.e., % homology=# of identical or similar positions/total # of positions×100). Alignment strategies are well known in the art; see, for example, Altschul et al. supra for optimal sequence alignment. MarA family polypeptides may share some amino acid sequence similarity with MarA. The nucleic acid and amino acid sequences of MarA as well as other MarA family polypeptides are available in the art. For example, the nucleic acid and amino acid sequence of MarA can be found, e.g., on GeneBank (accession number M96235) or in Cohen et al. 1993. J. Bacteriol. 175:1484. In one embodiment, a MarA family polypeptide excludes one or more of XyIS, AraC, and MeIR. In another embodiment, the MarA family polypeptide is involved in antibiotic resistance. In yet another embodiment, the MarA family polypeptide is selected from the group consisting of: MarA, RamA, AarP, Rob, SoxS, and PqrA.

Exemplary MarA family polypeptides are shown in Table 1, and at Prosite (PS00041) and include: AarP, Ada, AdaA, AdiY, AfrR, AggR, AppY, AraC, CafR, CeID, CfaD, CsvR, D90812, EnvY, ExsA, FapR, HrpB, InF, InvF, LcrF, LumQ, MarA, MeIR, MixE, MmsR, MsmR, OrfR, Orf_f375, PchR, PerA, PocR, PqrA, RafR, RamA, RhaR, RhaS, Rns, Rob, SoxS, 552856, TetD, TcpN, ThcR, TmbS, U73857, U34257, U21191, UreR, VirF, XyIR, XyIS, Xys1, 2, 3, 4, Ya52, YbbB, YfiF, YisR, YzbC, and YijO.

TABLE 1
Some Bacterial MarA homologs^{a}

Gram-negative bacteria
Escherichia coli
AfrR (1)
AggR (2)
AraC (3)
BfpT (PerA) (4)
CelD (5)
CfaD(CfaR) (6, 7)
CsvR (8)
D90812 (9)
FapR (10, 11)
MarA (12)
MelR (13)
ORF f375 (14, 15)
OrfR (16, 17)
RhaR (18, 19, 20)
RhaS (21)
Rns (22)
Rob (23)
SoxS (24, 25)
U73857 (26)
UreR (27)
XylR (28)
YijO (29)
Salmonella typhimurium
HilC(SirC) (30)
HilD (30)
InvF (30, 31)
MarA (32, 33)
PocR (34)
RamA (35)
Rob (33)
RtsA (30)
SoxS (33)
Acinetobacter baumanii
EsvA (36)
Brucella abortus
DhbR (37)
Citrobacter rodentium
RegA (38)
Providencia stuartii
AarP (39)
UreR (40)
Pseudomonas spp.
ChpD (41)
ExsA (42)
MmsR (43)
PA1229 (44)
PA1850 (45)
PchR (Michel 46)
TmbS (47)
VqsM (48)
XylS (49)
Xys1, 2, 3, 4 (50, 51)
Yersinia spp.
CafR (52)
LcrF (VirF) (53)
MarA47 (54)
RobA (55)
YbtA (56)
YsaE (57)
Shigella flexneri
MxiE (58)
VirF (58)
Vibrio cholerae
ToxT (59)
Proteus spp.
PqrA (60)
UreR (61)
Enterobacter aerogenes
MarA (62)
RamA (62)
Kiebsiella pneumoniae
RamA (63)
Burkholderia pseudomallei
BsaN (64)
Edwardsiella tarda
EsrC (65)
Haemophilus influenzae
Ya52 (66)
Gram-positive bacteria
Bordetella spp.
AlcR (67)
Staphylococcus aureus
Rbf (68)
Streptococcus mutans
MsmR (69)
Entercoccus faecalis
PerA (70)
Pediococcus pentosaceus
RafR (71)
Streptomyces spp.
U21191 (72)
AraL (73)
TxtR (74)
Bacillus subtilis
AdaA (75)
YbbB (76)
YfiF (77)
YisR (78)
YzbC (79)
Photobacterium leiognathi
LumQ (80)
Lactobacillus helveticus
U34257 (81)
Azorhizobium caulinodans
S52856 (82)
Cyanobacteria
Synechocystis spp.
LumQ (83)
PchR (83)
Other bacteria
Mycobacterium tuberculosis
AlkA (Rv1931c) 84)
VirS (Rv3082c) (85)
^{a}The smaller MarA homologs, ranging in size from 87 (U34257) to 138 (OrfR) amino acid residues, are represented in boldface. References are given in parentheses and are listed below.

### References for Table 1:

(1) M. K. Wolf, et al., 1990. Infect. Immun. 58:1124-1128.
(2) E. G. Dudley, et al., 2006. Mol. Micro. 61:1267-1282.
(3) C. M. Stoner, et al. 1982. J. Mol. Biol. 153:649-652.
(4) C. Bieber, et al., 1998. Science 280:2114-2118.
(5) L. L. Parker, et al., 1990. Genetics 123:455-471.
(6) M. C. Pilonieta, et al., 2007. J. Bacteriol. 189:5060-5067.
(7) J. Caron, et al., 1990. Infect. and Immun. 58:3442-3444.
(8) L. A. de Haan, et al., 1991. FEMS Microbiol. Lett. 67:341-346.
(9) H. Mori, 1996. Unpublished data taken from the NCBI databases.
(10) P. Klaasen, et al., 1990. Mol. Microbiol. 4:1779-1783.
(11) M. Ahmed, et al., 1994. J. Biol. Chem. 269-28506-28513.
(12) S. P. Cohen, et al. 1993. J. Bacteriol. 175:1484-1492.
(13) C. Webster, et al., 1989. Gene 83:207-213.
(14) G. Plunkett, III. 1995. Unpublished.
(15) C Garcia-Martin, et al., 1992. J. Gen. Microbiol. 138:1109-1116.
(16) G. M. Braus, et al. 1984. J. Bacteriol. 160:504-509.
(17) K. Schollmeier, et al., 1984. J. Bacteriol. 160:499-503.
(18) G. Plunkett, III., et al. 1993. Nucleic Acids Res. 21:3391-3398.
(19) C. G. Tate, et al. 1992. J. Biol. Chem. 267:6923-6932.
(20) J. F. Tobin et al., 1987. J. Mol. Biol. 196:789-799.
(21) J. Nishitani, 1991. Gene 105:37-42.
(22) M. C. Bordero, et al., 2008. BMC Microbiol. 8:180-190.
(23) R. E. Benz, et al., 1993. Zentralbl. Bakteriol. Parasitenkd. Infektionskr. Hyg. Abt. 1 Orig. 278:187-196.
(24) C. F. Amabile-Cuevas, et al., 1991. Nucleic Acids Res. 19:4479-4484.
(25) J. Wu, et al., 1991. J. Bacteriol. 173:2864-2871.
(26) M. Duncan, et al., 1996. Unpublished data.
(27) S. E. F. D'Orazio, et al., 1993. J. Bacteriol. 175:3459-3467.
(28) H. J. Sofia, et al., 1994. Nucleic Acids Res. 22:2576-2586.
(29) F. R. Blattner, et al., 1993. Nucleic Acids Res. 21:5408-5417.
(30) C. D. Ellermeier, et al., 2005. Mol. Micro. 57:691-705.
(31) K. Kaniga, et al., 1994. Mol. Microbiol. 13:555-568.
(32) M. C. Sulavik, et al., 1997. J. Bacteriol. 179:1857-1866.
(33) E. Hartog, et al., 2008. Microbiol. Immunol. 52:565-574.
(34) J. R. Roth, et al. 1993. J. Bacteriol. 175:3303-3316.
(35) T. van der Straaten, et al., 2004. Infect. and Immun. 72:996-1003.
(36) M. G. Smith, et al., 2007. Genes & Devel. 21: 601-614.
(37) E. S. Anderson, et al., 2008. J. Bacteriol. 190:1838-1842.
(38) E. Hart, et al., 2008. Infect. and Immun. 76:5247-5256.
(39) G. Cornelis, et al., 1989. J. Bacteriol. 171:254-262.
(40) I. Gendlina, et al., 2002. J. Biol. Chem. 277:37349-37358.
(41) C. B. Whitchurch, et al., 2004. Mol. Micro. 52:873-893.
(42) D. W. Frank, et al., 1991. J. Bacteriol. 173:6460-6468.
(43) D. R. Macing a, et al., 1995. J. Bacteriol. 177:3407-3413.
(44) M. C. Wolfgang, et al., 2003. Dev. Cell 4:253-263.
(45) M. J. Filiatrault, et al., 2006. Infect. and Immun. 74:4237-4245.
(46) L. Michel, et al., 2007. Microbiology 153:1508-1518.
(47) M. I. Steele, et al., 1992. J. Biol. Chem. 267:13585-13592.
(48) Y. H. Dong, et al., 2005. Mol. Micro. 58:552-564.
(49) G. Deho, et al., 1995. Unpublished data.
(50) N. Mermod, et al., 1984. EMBO J. 3:2461-2466.
(51) S. J. Assinder, et al., 1992. Nucleic Acids Res. 20:5476.
(52) E. E. Galyov, et al., 1991. FEBS Lett. 286:79-82.
(53) N. P. Hoe, et al., 1992. J. Bacteriol. 174:4275-4286.
(54) R. A. Udani, et al., 2006. Antimicrob. Agents Chemother. 50:2971-2975.
(55) K. L. Stirrett, et al., 2008. BMC Microbiol. 8:122-133.
(56) H. Gao, et al., 2008. J. Bacteriol. 190:3063-3075.
(57) K. A. Walker, et al., 2004. J. Bacteriol. 186:4056-4066.
(58) M. Mavris, et al., 2002. Mol. Micro. 43:1543-1553.
(59) D. T. Hung, et al., 2005. Science 310:670-674.
(60) H. Ishida, et al., 1995. Antimicrob. Agents Chemother. 39:453-457.
(61) J. D. Dattlebaum, et al., 2003. Infect. and Immun. 71:1026-1030.
(62) R. Chollet, et al., 2004. Antimicrob. Agents Chemother. 48:2518-2523.
(63) A. M. George, et al., 1983. J. Bacteriol. 155:541-548.
(64) M. P. Stevens, et al., 2002. Mol. Micro. 46:649-659.
(65) J. Zheng, et al., 2005. Infect. and Immun. 73:4127-4137.
(66) R. D. Fleischmann, et al., 1995. Science 269:469-512.
(67) F. C. Beaumont, et al., 1998. J. Bacteriol. 180:862-870.
(68) Y. Lim, et al., 2004. J. Bacteriol. 186:722-729.
(69) R. R. Russell, et al., 1992. J. Biol. Chem., 267:4631-4637.
(70) P. S. Coburn, et al., 2008. Infect. and Immun. 76:5668-5676.
(71) K. K. Leenhouts, et al., 1995. Unpublished data.
(72) J. Kormanec, et al., 1995. Gene 165:77-80.
(73) C. W. Chen, et al., 1992. J. Bacteriol. 174:7762-7769.
(74) M. V. Joshi, et al., 2007. Mol. Micro. 66:633-642.
(75) F. Morohoshi, et al. 1990. Nucleic Acids Res. 18:5473-5480.
(76) M. Rosenberg, et al., 1979. Annu. Rev. Genet. 13:319-353.
(77) H. Yamamoto, et al., 1996. Microbiology 142:1417-1421.
(78) L. B. Bussey, et al., 1993. J. Bacteriol. 175:6348-6353.
(79) P. G. Quirk, et al., 1994. Biochim. Biophys. Acta 1186:27-34.
(80) J. W. Lin, et al., 1995. Biochem. Biophys. Res. Commun. 217:684-695.
(81) E. G. Dudley, et al., 1996. J. Bacteriol. 178:701-704.
(82) D. Geelen, et al., 1995. Unpublished data.
(83) S. J. Assinder, et al., 1993. J. Gen. Microbiol. 139:557-568.
(84) C. C. Frota, et al., 2004. Infect. and Immun. 72:5483-5486.
(85) A. Singh, et al., 2005. J. Bacteriol. 187:4173-4186.

The term “transcription factor modulating compound” or “transcription factor modulator” includes compounds that interact with one or more transcription factors, such that the activity of the transcription factor is modulated, e.g., enhanced or inhibited. The term also includes both AraC family modulating compounds and MarA family modulating compounds (e.g., compounds that modulate transcription factors of the AraC family and compounds that modulate transcription factors of the MarA family, respectively). In another embodiment, the transcription factor modulating compound is a compound that inhibits a transcription factor, e.g., a prokaryotic transcription factor or a eukaryotic transcription activation factor. In one embodiment, the transcription factor modulating compounds modulate the activity of a transcription factor as measured by assays known in the art or LANCE assays such as those described in Example 12. In one embodiment, the transcription factor modulating compound inhibits the binding of a particular transcription factor to its cognate DNA by about 10% or greater, about 40% or greater, about 50% or greater, about 60% or greater, about 70% or greater, about 80% or greater, about 90% or greater, about 95% or greater, or about 100% as compared to the activity in the absence of the transcription factor modulating compound.

In another embodiment, the transcription factor modulating compound is a MarR family polypeptide inhibitor. In another embodiment, the transcription factor modulating compound is a AraC family polypeptide inhibitor.

The invention also pertains to a method for preventing bacterial growth on a contact lens. The method includes contacting the contact lenses with a solution containing a transcription factor modulating compound, e.g., a compound of formula I, II, III, IV, IVa, VI, VIa, VII, or VIII or a compound of Table 2, in an acceptable carrier. The invention also pertains to a solution comprising the compound, packaged with directions for using the solution to clean contact lenses.

In yet another embodiment, the invention pertains, at least in part, to a method for the prevention or treatment of an infection in a patient into which an indwelling device has been implanted by administering to the patient a composition comprising a transcription factor modulating compound, e.g., a compound of formula I, II, III, IV, IVa, VI, VIa, VII, or VIII or a compound of Table 2. The method includes contacting at least one compound of the invention with a medical indwelling device, such as to prevent or substantially inhibit the formation of a biofilm. Examples of medical indwelling devices include catheters, orthopedic devices, devices associated with endotracheal intubation, devices associated with mechanical ventilation (e.g., a ventilator) and implants.

In one embodiment, the invention pertains, at least in part, to a method for treating or preventing biofilm formation or a biofilm associated state in a subject by administering to the subject an effective amount of a transcription factor modulating compound, e.g., a compound of formula I, II, III, IV, IVa, VI, VIa, VII, or VIII or a compound of Table 2. The biofilm associated state includes disorders which are characterized by the presence or potential presence of a bacterial biofilm and can include, for example, middle ear infections, cystic fibrosis, osteomyelitis, acne, dental cavities, endocarditis, pneumonia and prostatitis. Biofilm is also implicated with, e.g., Pseudomonas aeruginosa. Furthermore, the invention also pertains to methods for preventing the formation of biofilms on surfaces or in areas by contacting the area with an effective amount of a transcription factor modulating compound, e.g., a MarA family inhibiting compound, etc. In one embodiment, the biofilm associated state is ventilator associated pneumonia. In yet another embodiment, the invention pertains, at least in part to a method for treating or preventing pneumonia in a subject where the pneumonia is associated with Pseudomonas aeruginosa.

In another embodiment, the transcription factor modulating compound inhibits biofilm formation, for example, as measured by assays known in the art or the Crystal Violet assay described in Example 11. In one embodiment, the transcription factor modulating compound of the invention inhibits the formation of a biofilm by about 25% or more, 50% or more, 75% or more, 80% or more, 90% or more, 95% or more, 96% or more, 97% or more, 98% or more, 99% or more, 99.9% or more, 99.99% or more, or by 100%, as compared to the formation of a biofilm without the transcription factor modulating compound.

The term “biofilm” includes biological films that develop and persist at interfaces in aqueous and other environments. Biofilms are composed of microorganisms embedded in an organic gelatinous structure composed of one or more matrix polymers which are secreted by the resident microorganisms. The term “biofilm” also includes bacteria that are attached to a surface in sufficient numbers to be detected or communities of microorganisms attached to a surface (Costerton, J. W., et al. 1987. Ann. Rev. Microbiol. 41:435-464; Shapiro, J. A. 1988. Sci Am. 256:82-89; O'Toole, G. et al. 2000. Ann. Rev. Microbiol. 54:49-79).

In a further embodiment, the invention pertains, at least in part to a method for preventing or treating a bacterial infection in a subject by administering to the subject an effective amount of a transcription factor modulating compound, e.g., a compound of formula I, II, III, IV, IVa, VI, VIa, VII, or VIII or a compound of Table 2. The term “bacterial infection” includes states characterized by the presence of bacteria which can be prevented or treated by administering the transcription factor modulating compounds of the invention. The term includes biofilm formation and other infections or the undesirable presence of a bacteria on or in a subject. In one embodiment, the bacterial infection is associated with Y. pseudotuberculosis, Y. pestis or P. aeruginosa. In yet another embodiment, the bacterial infection is associated with burn wounds or corneal ulcers. In another embodiment, the bacterial infection is associated with the implantation of a medical device in a subject (e.g., in the case of mechanical ventilation, endotracheal intubation, catheterization, and the like). In a further embodiment, the bacterial infection is a nosocomial infection.

In a further embodiment, the invention pertains, at least in part, to a method of treating or preventing pneumonia (e.g., ventilator-associated pneumonia) in a subject by administering to the subject an effective amount of a transcription factor modulating compound. In another embodiment, the invention pertains, at least in part, to a method of inhibiting a MarA family polypeptide by contacting a MarA family polypeptide with an effective amount of a transcription factor modulating compound. Suitable MarA family polypeptides include, but are not limited to, ExsA, LcrF (VirF) or Sox.

In one embodiment, the invention pertains, at least in part, to a method of treating or preventing burn wounds or corneal ulcers in a subject by administering to the subject an effective amount of a transcription factor modulating compound, e.g., a compound of formula I, II, III, IV, IVa, VI, VIa, VII, or VIII or a compound of Table 2.

In yet another embodiment, the invention pertains, at least in part, to a method for treating or preventing a urinary tract infection in a subject by administering to the subject an effective amount of a transcription factor modulating compound, e.g., a compound of formula I, II, III, IV, IVa, VI, VIa, VII, or VIII or a compound of Table 2.

In another embodiment, the invention pertains, at least in part, to a method for treating or preventing of a kidney infection in a subject by administering to the subject an effective amount of a transcription factor modulating compound, e.g., a compound of formula I, II, III, IV, IVa, VI, VIa, VII, or VIII or a compound of Table 2.

In an embodiment, the invention pertains, at least in part, to a method for treating or preventing acute pyelonephritis in a subject by administering to the subject an effective amount of a transcription factor modulating compound, e.g., a compound of formula I, II, III, IV, IVa, VI, VIa, VII, or VIII or a compound of Table 2.

In one embodiment, the invention pertains, at least in part, to a method of inhibiting bacterial infectivity and/or virulence of a bacteria by administering to a subject suffering from or at risk of suffering from a bacterial infection an effective amount of a transcription factor modulating compound of the invention, e.g., a compound of formula I, II, III, IV, IVa, VI, VIa, VII, or VIII or a compound of Table 2.

In one embodiment, the invention pertains to a method of treating or preventing an infection in a subject by administering an effective amount of a transcription factor modulating compound of the invention, e.g., a compound of formula I, II, III, IV, IVa, VI, VIa, VII, or VIII or a compound of Table 2, to the subject. The aforementioned infection includes, but is not limited to, an infection by Staphylococcus aureus, Enterococcus faecium, Streptococcus pyogenes, Streptococcus pneumoniae, Streptococcus pneumoniae, Y. pseudotuberculosis, Y. pestis or P. aeruginosa.

In another embodiment, the present invention pertains, at least in part, to a method for modulating transcription of genes regulated by transcription factors in the MarA (AraC) family by contacting a transcription factor with a transcription factor modulating compound of the invention, e.g., a compound of formula I, II, III, IV, IVa, VI, VIa, VII, or VIII or a compound of Table 2. In one embodiment, the member of the MarA (AraC) family is ExsA or VirF.

In one embodiment, the transcription factor modulating compound is a compound of formula I:

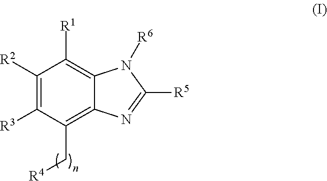

wherein

R¹, R², R³, and R⁵ are each independently hydrogen, hydroxyl, alkyl, alkenyl, alkynyl, aryl, amino, sulfonyl, carbonyl, carboxy, alkoxy, aryloxy, halogen, acyl, oximyl, hydrazinyl, —NO₂, —CN, a heterocyclic moiety or thioether,

R⁴ is —COOR⁷, —SO₃R⁸ or —PO(OR⁹)₂;

R⁶ is hydrogen, hydroxy, alkoxy, alkyl, alkenyl, alkynyl, aryl, carbonyl, carboxy, acyl or NR¹⁰R¹¹;

R⁷ is hydrogen, alkyl, alkenyl, alkynyl, aryl, a heterocyclic moiety or carbonyl;

R⁸ and R⁹ are each independently hydrogen, alkyl, alkenyl, alkynyl or aryl;

R¹⁰ and R¹¹ are each independently hydrogen, alkyl, alkenyl, alkynyl, aryl, a heterocyclic moiety or carbonyl; and

n is an integer of between 1 and 10; and pharmaceutically acceptable salts thereof.

In one embodiment, R⁶ is hydroxyl, R¹ and R³ are hydrogen, R² is —NO₂, n is 1, R⁴ is —COOR⁷, R⁷ is hydrogen, R⁵ is aryl (e.g., substituted phenyl, for example, para substituted phenyl). In one embodiment, the substituted phenyl is substituted with a moiety of formula (Ia):

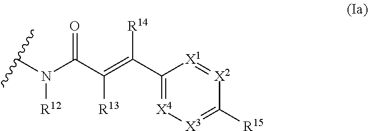

wherein

R¹² is hydrogen, hydroxyl, alkoxy, alkyl, alkenyl, alkynyl, aryl, a heterocyclic moiety or carbonyl;

R¹³, R¹⁴ and R¹⁵ are each independently hydrogen, hydroxyl, alkyl, alkenyl, alkynyl, aryl, amino, thioether, sulfonyl, carbonyl, a heterocyclic moiety, carboxy, alkoxy, aryloxy, halogen or acyl;

X¹, X², X³ and X⁴ are each independently nitrogen or CR¹⁶; and

R¹⁶ is hydrogen, hydroxyl, alkyl, alkenyl, alkynyl, aryl, amino, acyl, carbonyl, carboxy, alkoxy, aryloxy, halogen, a heterocyclic moiety or acyl.

In a further embodiment, R¹², R¹³ and R¹⁴ are each hydrogen, X¹, X², X³ and X⁴ are each CR¹⁶, R¹⁶ is hydrogen and R¹⁵ is acyl or halogen (e.g., fluorine).

In another embodiment, X¹, X² and X⁴ are each CR¹⁶ and X³ is nitrogen, R¹⁶ is hydrogen and R¹⁵ is acyl.

In one embodiment, R⁶ is hydroxyl, R¹ and R³ are hydrogen, R² is —NO₂, n is 1, R⁴ is —COOR⁷, R⁷ is hydrogen, R⁵ is aryl (e.g., substituted phenyl, for example, para substituted phenyl). In one embodiment, the substituted phenyl may be substituted with a moiety of formula (Ib):

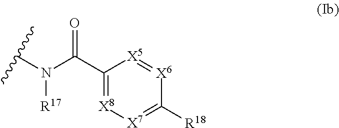

wherein

R¹⁷ is hydrogen, hydroxyl, alkoxy, alkyl, alkenyl, alkynyl, aryl, a heterocyclic moiety or carbonyl;

R¹⁸ is hydrogen, hydroxyl, alkyl, alkenyl, alkynyl, aryl, amino, thioether, sulfonyl, carbonyl, carboxy, alkoxy, aryloxy, halogen, a heterocyclic moiety or acyl;

X⁵, X⁶ X⁷ and X⁸ are each independently nitrogen or CR¹⁹; and

R¹⁹ is hydrogen, hydroxyl, alkyl, alkenyl, alkynyl, aryl, amino, acyl, carbonyl, a heterocyclic moiety, carboxy, alkoxy, aryloxy, halogen or acyl.

In another embodiment, R¹⁷ is hydrogen, X⁵, X⁶, X⁷ and X⁸ are each CR¹⁹, R¹⁹ is hydrogen and R¹⁸ is acyl or aryl (e.g., heteroaryl, for example, imidazole).

In a further embodiment, X⁵, X⁶ and X⁸ are each CR¹⁹ and X⁷ is nitrogen, R¹⁹ is hydrogen and R¹⁸ is a heterocyclic moiety (e.g., morpholinyl).

In one embodiment, the transcription factor modulating compound is a compound of formula (II):

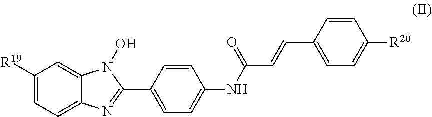

wherein

R¹⁹ is —COOH, —Cl, —NO₂, —CN, —SCH₃, —COCH₃, —F, —SO₂CH₃, —H or —CF₃; and

R²⁰ is —COCH₃, —SCH₃ or —C(OH)₂CF₃ and pharmaceutically acceptable salts thereof.

In one embodiment, R²⁰ is —SCH₃ and R¹⁹ is —NO₂ or —CN.

In another embodiment, R²⁰ is —C(OH)₂CF₃ and R¹⁹ is —NO₂ or —CN.

In yet another embodiment, R²⁰ is —F and R¹⁹ is —COCH₃. F, —SO₂CH₃, —H, —CF₃, Cl or COOH.

In a further embodiment, R²⁰ is —COCH₃ and R¹⁹ is —Cl.

In one embodiment, when R²⁰ is —COCH₃, R¹⁹ is not —NO₂, —F, —CN or —COCH₃.

In another embodiment, the transcription factor modulating compound is a compound of formula (III):

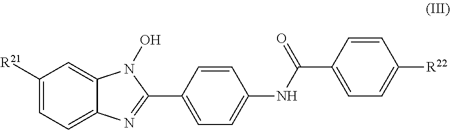

wherein

R²¹ is —NO₂ or —CN; and

R²² is —C(OH)₂CF₃ and pharmaceutically acceptable salts thereof.

In one embodiment, R²¹ is —NO₂. In another embodiment, R²² is —CN.

In yet another embodiment, the transcription factor modulating compound is a compound of formula (IV):

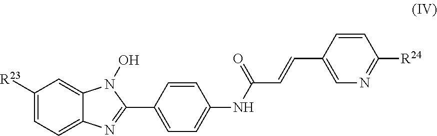

wherein

R²³ and R²⁴ are each independently hydrogen, hydroxyl, alkyl, alkenyl, alkynyl, aryl, amino, sulfonyl, carbonyl, carboxy, alkoxy, aryloxy, halogen, acyl, oximyl, hydrazinyl, —NO₂, —CN, a heterocyclic moiety or thioether, and pharmaceutically acceptable salts thereof.

In one embodiment, R²⁴ is a thioether (e.g., —SCH₃) and R²³ is —NO₂ or —CN.

In another embodiment, R²⁴ is amino (e.g., dialkylamino, such as dimethylamino) and R²³ is —NO₂.

In yet another embodiment, R²⁴ is acyl (e.g., —COCH₃) and R²³ is —CN.

In one embodiment, when R²³ is —NO₂, then R²⁴ is not —CF₃, —COCH₃ or imidazole.

In a further embodiment, when R²³ is —CN, then R²⁴ is not imidazole.

In another embodiment, the compound of formula (IV) is of formula (IVa):

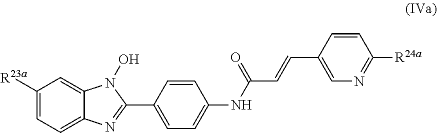

wherein

R^{23a} is —CN or —NO₂; and

R^{24a} is acyl, thioether or amino, and pharmaceutically acceptable salts thereof.

In yet another embodiment, the transcription factor modulating compound is a compound of formula (VI):

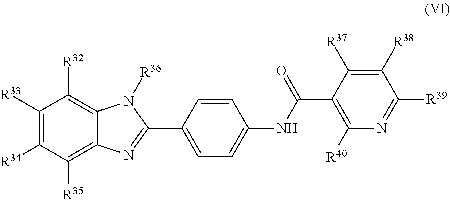

wherein

R³², R³³, R³⁴, R³⁵, R³⁶, R³⁷, R³⁸, R³⁹ and R⁴⁰ are each independently hydrogen, hydroxyl, alkyl, alkenyl, alkynyl, aryl, amino, sulfonyl, carbonyl, carboxy, alkoxy, aryloxy, halogen, acyl, oximyl, hydrazinyl, —NO₂, —CN, a heterocyclic moiety or thioether;

R³⁶ is hydrogen, hydroxy, alkoxy, alkyl, alkenyl, alkynyl, aryl, carbonyl, carboxy, acyl or NR^{36a}R^{36b};

R^{36a} and R^{36b} are each independently hydrogen, alkyl, alkenyl, alkynyl, aryl, a heterocyclic moiety or carbonyl, and pharmaceutically acceptable salts thereof.

In one embodiment, R³², R³⁴, R³⁵, R³⁷, R³⁸ and R⁴⁰ are each hydrogen, R³⁶ is hydroxyl, R³⁹ is aryl (e.g., pyrazole) and R³³ is —NO₂ or —CN.

In another embodiment, R³², R³⁴, R³⁵, R³⁷, R³⁸ and R⁴⁰ are each hydrogen, R³⁶ is hydroxyl, R³³ is —NO₂, R³⁹ is a heterocyclic moiety (e.g., N-methylpiperazine or piperidine), alkyoxy (e.g., —OCH₂CF₃) or amino (e.g., dialkylamino, such as dimethylamino).

In yet another embodiment, R³², R³⁴, R³⁵, R³⁷, R³⁸ and R⁴⁰ are each hydrogen, R³⁶ is hydroxyl, R³³ is —CN and R³⁹ is a heterocyclic moiety (e.g., morpholine).

In one embodiment, R³², R³⁴, R³⁵, R³⁷ and R³⁸ are each hydrogen, R³⁶ is hydroxyl, R³⁹ is alkyl (e.g., trifluormethyl) and R⁴⁰ is alkyl (e.g., methyl).

In another embodiment, when R³², R³⁴, R³⁵, R³⁷, R³⁸ and R⁴⁰ are each hydrogen, R³³ is —NO₂ and R³⁶ is —OH, then R³⁹ is not hydrogen or morpholine.

In another embodiment, R³⁷, R³⁸, R³⁹ and R⁴⁰ are not all hydrogen.

In yet another embodiment, the compound of formula (VI) is of formula (VIa):

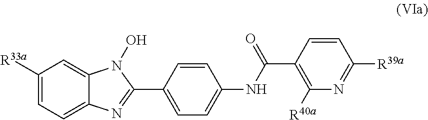

wherein

R^{33a} is —NO₂ or —CN;

R^{39a} is aryl, alkyl, a heterocyclic moiety, alkyoxy or amino; and

R^{40a} is hydrogen or alkyl, and pharmaceutically acceptable salts thereof.

In yet another embodiment, the transcription factor modulating compound is a compound of formula (VII):

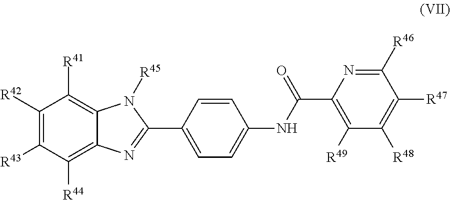

wherein

R⁴¹, R⁴², R⁴³, R⁴⁴, R⁴⁶, R⁴⁷, R⁴⁸ and R⁴⁹ are each independently hydrogen, hydroxyl, alkyl, alkenyl, alkynyl, aryl, amino, sulfonyl, carbonyl, carboxy, alkoxy, aryloxy, halogen, acyl, oximyl, hydrazinyl, —NO₂, —CN, a heterocyclic moiety or thioether;

R⁴⁵ is hydrogen, hydroxy, alkoxy, alkyl, alkenyl, alkynyl, aryl, carbonyl, carboxy, acyl or NR^{45a}R^{45b};

R^{45a} and R^{45b} are each independently hydrogen, alkyl, alkenyl, alkynyl, aryl, a heterocyclic moiety or carbonyl, and pharmaceutically acceptable salts thereof.

In one embodiment, R⁴¹, R⁴³, R⁴⁴, R⁴⁷, R⁴⁸ and R⁴⁹ are each hydrogen, R⁴² is —NO₂, R⁴⁵ is hydroxyl and R⁴⁶ is a heterocyclic moiety (e.g., morpholine).

In a further embodiment, the transcription factor modulating compound is a compound of formula (VIII):

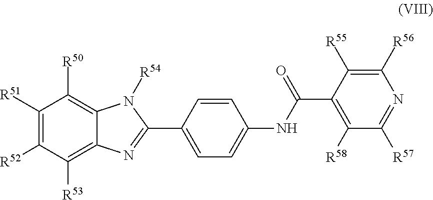

wherein

R⁵⁰, R⁵¹, R⁵², R⁵³, R⁵⁵, R⁵⁶, R⁵⁷ and R⁵⁹ are each independently hydrogen, hydroxyl, alkyl, alkenyl, alkynyl, aryl, amino, sulfonyl, carbonyl, carboxy, alkoxy, aryloxy, halogen, acyl, oximyl, hydrazinyl, —NO₂, —CN, a heterocyclic moiety or thioether;

R⁵⁴ is hydrogen, hydroxy, alkoxy, alkyl, alkenyl, alkynyl, aryl, carbonyl, carboxy, acyl or NR^{54a}R^{54b};

R^{54a} and R^{54b} are each independently hydrogen, alkyl, alkenyl, alkynyl, aryl, a heterocyclic moiety or carbonyl, and pharmaceutically acceptable salts thereof.

In one embodiment, R⁵⁰, R⁵², R⁵³, R⁵⁵, R⁵⁷ and R⁵⁸ are each hydrogen, R⁵¹ is —NO₂, R⁵⁴ is hydroxyl and R⁵⁶ is a heterocyclic moiety (e.g., morpholine).

In another embodiment, when R⁵⁰, R⁵², R⁵³, R⁵⁵, R⁵⁷ and R⁵⁸ are each hydrogen, R⁵¹ is —NO₂ and R⁵⁴ is hydrogen, then R⁵⁶ is not hydrogen.

In one embodiment, the transcription factor modulating compound is a compound of Table 2, or a pharmaceutically acceptable salt thereof:

TABLE 2

| | |
|---|---|
| A | 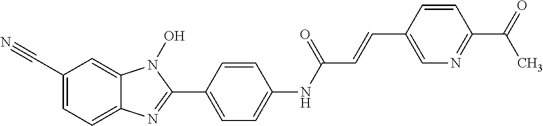 |
| B | 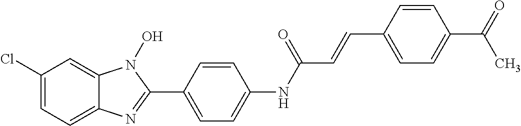 |
| C | 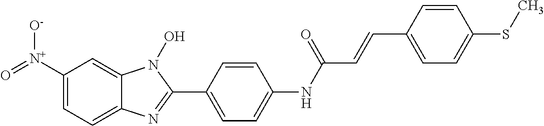 |
| D | 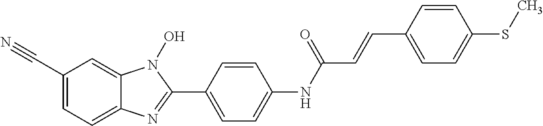 |
| E | 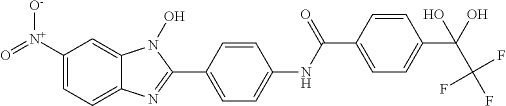 |
| F | 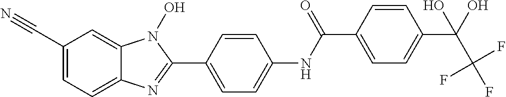 |
| G | 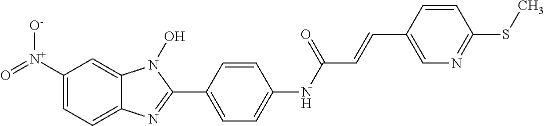 |
| H | 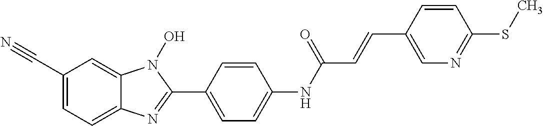 |
| I | 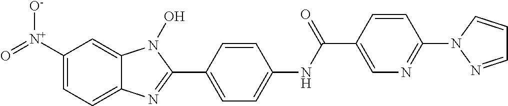 |
| J | 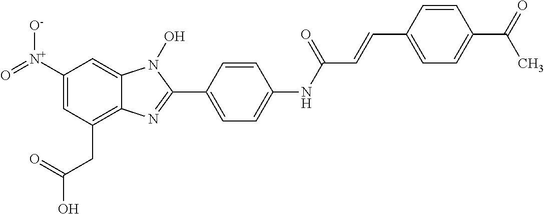 |
| K | 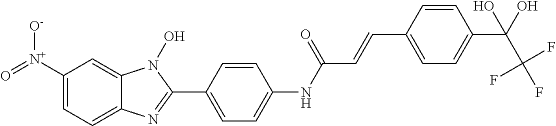 |
| L | 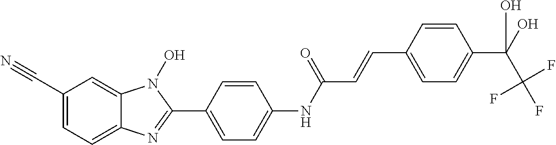 |
| M | 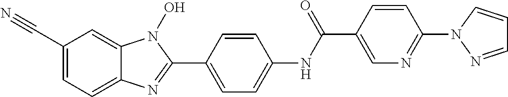 |
| N | 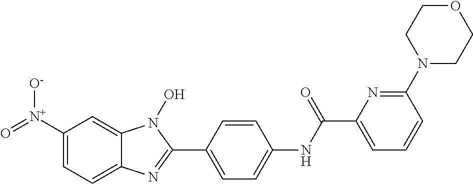 |
| O | 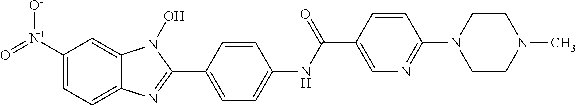 |
| P | 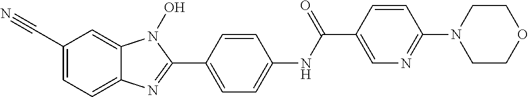 |
| Q | 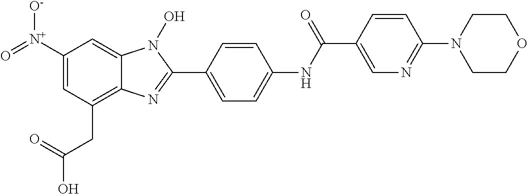 |
| R | 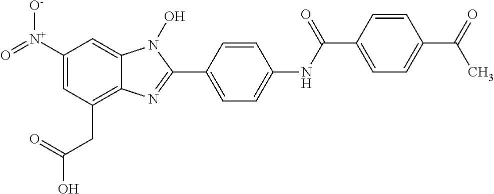 |
| S | 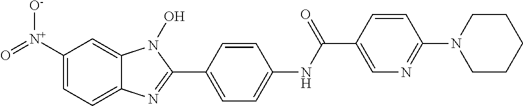 |
| T | 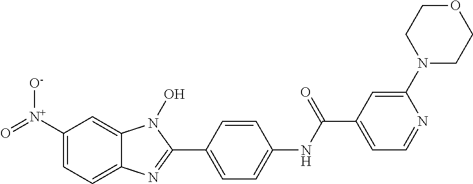 |
| U | 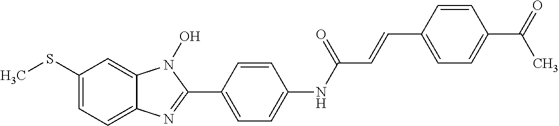 |
| V | 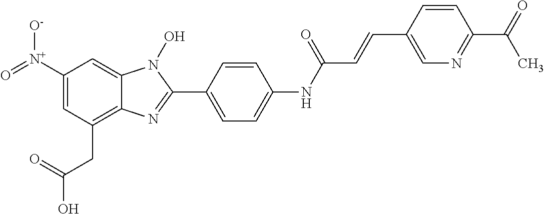 |
| W | 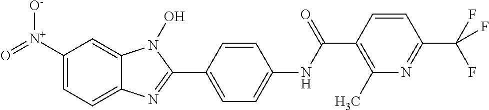 |
| X | 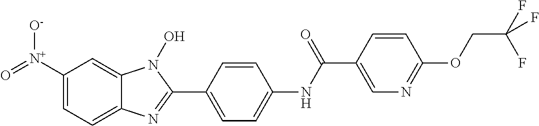 |
| Y | 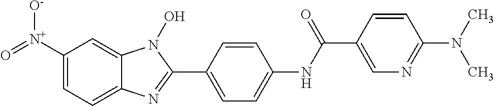 |
| Z | 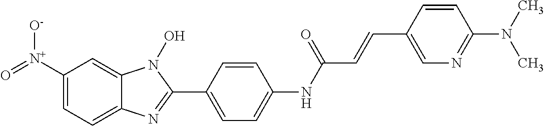 |
| AA | 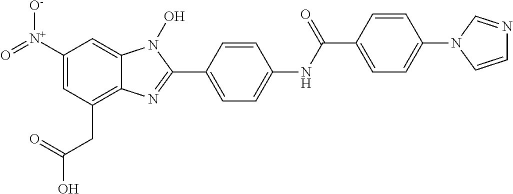 |
| AB | 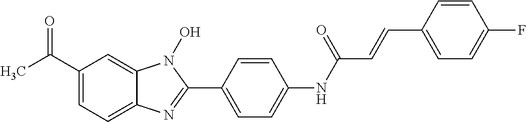 |
| AC | 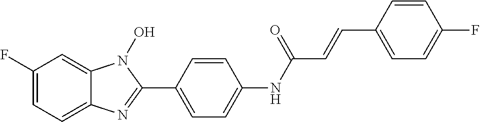 |
| AD | 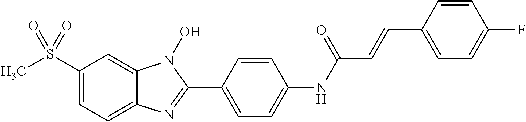 |
| AE | 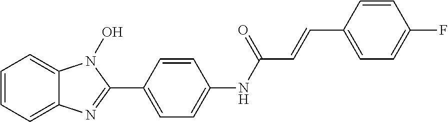 |
| AF | 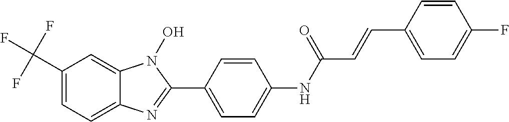 |
| AG | 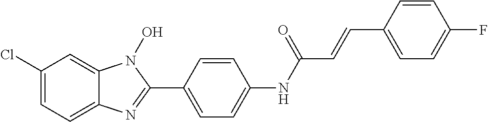 |
| AH | 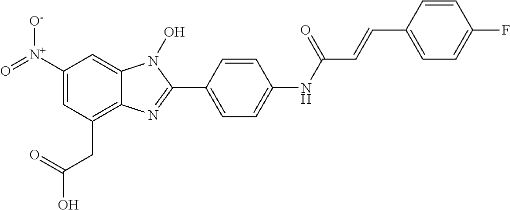 |
| AI | 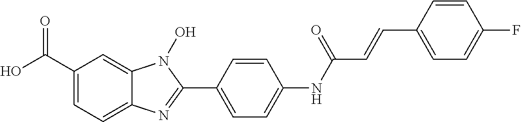 |

In one embodiment, the pharmaceutically acceptable salt is sodium or potassium.

In one embodiment, the transcription factor modulating compounds of the present invention do not include compounds disclosed in U.S. Pat. No. 7,405,235; U.S. patent application Ser. No. 12/069,723; U.S. patent application Ser. No. 11/115,024; U.S. patent application Ser. No. 11/823,103 and U.S. patent application Ser. No. 12/057,357.

The IC₅₀ of a transcription factor modulating compound can be measured using the assay described in Example 2. In a further embodiment, the transcription factor modulating compound has an IC₅₀ activity against SoxS of less than about 10 μM, less than about 5 μM, or less than about 1 μM, as described in Example 10. In a further embodiment, the transcription factor modulating compound can have an IC₅₀ activity against MarA of less than about 10 μM, less than about 5 μM, or less than about 1 μM. In yet another embodiment, the transcription factor modulating compound can have an IC₅₀ against LcrF (VirF) of less than about 10 μM, less than about 5 μM, or less than about 1 μM, as described in Example 4. In a further embodiment, the transcription factor modulating compound can have an IC₅₀ against ExsA of less than about 10 μM, less than about 5 μM, or less than about 1 μM, as described in Example 7.

In one embodiment, the invention pertains, at least in part, to a method for reducing or preventing the spread of microbial cells from one or more organs (e.g., liver, kidney, lungs, brain or spleen) to another organ or organs in a subject by administering to the subject an effective amount of a transcription factor modulating compound (e.g., a compound of formula I, II, III, IV, IVa, VI, VIa, VII, or VIII or a compound of Table 2). In another embodiment, the invention pertains, at least in part, to a method for reducing the bacterial burden (e.g., the amount of bacteria) in one or more organs in the subject's body (e.g., lungs, brain, liver, spleen and kidneys) by administering an effective amount of a transcription factor modulating compound (e.g., a compound of formula I, II, III, IV, IVa, VI, VIa, VII, or VIII or a compound of Table 2). In another embodiment, the transcription factor modulating compound causes a log decrease in CFU/g of a tissue in an animal compared to control tissue, for example, in lung tissue or kidney tissue. This can be measured using the assay described Example 6. In one embodiment, the transcription factor modulating compound causes a log decrease in CFU/g of tissue of greater than 1.0 CFU/g. In a further embodiment, the compound causes a log decrease in CFU/g of tissue greater than 2.5 CFU/g.

In another embodiment, the transcription factor modulating compound (e.g., a compound of formula I, II, III, IV, IVa, VI, VIa, VII, or VIII or a compound of Table 2) induces a decrease in the cytotoxicity of a microbial agent (e.g., the ability of a microbial agent to kill a cell). In one embodiment, the transcription factor modulating compound inhibits the cytotoxicity of a microbe compared to a control, as described in Examples 5 and 8. In one embodiment, the cytotoxicity is inhibited by about 10%, by about 20%, by about 30%, about 40%, by about 50%, by about 60%, by about 70%, by about 80%, by about 90% or about 100%.

The term “alkyl” includes saturated aliphatic groups, including straight-chain alkyl groups (e.g., methyl, ethyl, propyl, butyl, pentyl, hexyl, heptyl, octyl, nonyl, decyl, etc.), branched-chain alkyl groups (isopropyl, tert-butyl, isobutyl, etc.), cycloalkyl (alicyclic) groups (cyclopropyl, cyclobutyl, cyclopentyl, cyclohexyl, cycloheptyl, cyclooctyl), alkyl substituted cycloalkyl groups, and cycloalkyl substituted alkyl groups. The term alkyl further includes alkyl groups that may include oxygen, nitrogen, sulfur or phosphorous atoms replacing one or more carbons of the hydrocarbon backbone. In certain embodiments, a straight chain or branched chain alkyl has 6 or fewer carbon atoms in its backbone (e.g., C₁-C₆ for straight chain, C₃-C₆ for branched chain). Likewise, cycloalkyls may have from 3-8 carbon atoms in their ring structure. The term “C₁-C₆” includes alkyl groups containing 1 to 6 carbon atoms.

Moreover, the term alkyl includes both “unsubstituted alkyls” and “substituted alkyls,” the latter of which refers to alkyl moieties having substituents replacing a hydrogen on one or more carbons of the hydrocarbon backbone. Such substituents can include, for example, alkyl, alkenyl, alkynyl, halogen, hydroxyl, aryl, alkylcarbonyloxy, arylcarbonyloxy, alkoxycarbonyloxy, aryloxycarbonyloxy, —COOH, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonate, sulfamoyl, sulfonamido, nitro, trifluoromethyl, cyano, azido, heterocyclyl, alkylaryl, or an aromatic or heteroaromatic moiety. Cycloalkyls can be further substituted, e.g., with the substituents described above. An “alkylaryl” or an “arylalkyl” moiety is an alkyl substituted with an aryl (e.g., phenylmethyl (benzyl)). The term “alkyl” also includes the side chains of natural and unnatural amino acids.

The term “aryl” includes groups, e.g., 5- and 6-membered single-ring aromatic groups, that may include from zero to four heteroatoms, for example, benzene, phenyl, pyrrole, furan, thiophene, thiazole, isothiaozole, imidazole, triazole, tetrazole, pyrazole, oxazole, isooxazole, pyridine, pyrazine, pyridazine, and pyrimidine, and the like. Furthermore, the term “aryl” includes multicyclic aryl groups, e.g., tricyclic, bicyclic, e.g., naphthalene, benzoxazole, benzodioxazole, benzothiazole, benzoimidazole, benzothiophene, methylenedioxyphenyl, quinoline, isoquinoline, napthridine, indole, benzofuran, purine, benzofuran, deazapurine, or indolizine. Those aryl groups having heteroatoms in the ring structure may also be referred to as “aryl heterocycles,” “heteroaryls” or “heteroaromatics.” The aromatic ring can be substituted at one or more ring positions with such substituents as described above, as for example, alkyl, alkenyl, alkynyl, halogen, hydroxyl, alkoxy, aryl, alkylcarbonyloxy, arylcarbonyloxy, alkoxycarbonyloxy, aryloxycarbonyloxy, —COOH, alkylcarbonyl, alkylaminoacarbonyl, arylalkyl aminocarbonyl, alkenylaminocarbonyl, alkylcarbonyl, arylcarbonyl, arylalkylcarbonyl, alkenylcarbonyl, alkoxycarbonyl, aminocarbonyl, alkylthiocarbonyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Aryl groups can also be fused or bridged with alicyclic or heterocyclic rings which are not aromatic so as to form a polycycle (e.g., tetralin). The term heteroaryl includes unsaturated cyclic compounds such as azirine, oxirene, dithiete, pyrroline, pyrrole, furan, dihydrofuran, dihydrothiophene, thiophene, pyrazole, imidazole, oxazole, thiazole, isothiazole, 12,2,3-triazole, 1,2,4, triazole, dithiazole, tetrazole, pyridine, pyran, pyrimidine, pyran, thiapyrane, diazine, thiazine, dioxine, triazine and tetrazene.

The term “heterocyclic moiety” includes saturated cyclic moieties having a closed ring of atoms in which at least one atom is not a carbon. As used herein, heterocyclic moieties do not include heteroaryl moieties, in which the closed ring of atoms is both heterocyclic and aromatic and/or unsaturated. Examples of heterocyclic moieties include aziridine, ethylene oxide, thiirane, dioxirane, azetidine, oxetane, thietane, dioxetane, dithietane, pyrrolidine, tetrahydrofuran, tetrahydrothiophene, imidazolidine, oxazolidine, thiazolidine, dioxolane, dithiolane, piperidine, tetrahydropyran, thiane, piperzine, pyrazine, dithiane, dioxane and trioxane.

The term “heterocyclic moiety” includes both “unsubstituted heterocyclic moieties” and “substituted heterocyclic moieties,” the latter of which includes moieties having substituents replacing a hydrogen on one or more of the atoms on the closed ring. Such substituents can include, for example, alkyl, alkenyl, alkynyl, halogens, hydroxyl, aryl alkylcarbonyloxy, arylcarbonyloxy, alkoxycarbonyl oxy, aryloxycarbonyloxy, —COOH, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “alkenyl” includes unsaturated aliphatic groups analogous in length and possible substitution to the alkyls described above, but that contain at least one double bond. For example, the term “alkenyl” includes straight-chain alkenyl groups (e.g., ethylenyl, propenyl, butenyl, pentenyl, hexenyl, heptenyl, octenyl, nonenyl, decenyl, etc.), branched-chain alkenyl groups, cycloalkenyl (alicyclic) groups (cyclopropenyl, cyclobutenyl, cyclopentenyl, cyclohexenyl, cycloheptenyl, cyclooctenyl), alkyl or alkenyl substituted cycloalkenyl groups, and cycloalkyl or cycloalkenyl substituted alkenyl groups. The term “alkenyl” further includes alkenyl groups which include oxygen, nitrogen, sulfur or phosphorous atoms replacing one or more carbons of the hydrocarbon backbone. In certain embodiments, a straight chain or branched chain alkenyl group has 6 or fewer carbon atoms in its backbone (e.g., C₂-C₆ or straight chain, C₃-C₆ for branched chain). Likewise, cycloalkenyl groups may have from 3-8 carbon atoms in their ring structure. The term C₂-C₆ includes alkenyl groups containing 2 to 6 carbon atoms.

Moreover, the term “alkenyl” includes both “unsubstituted alkenyls” and “substituted alkenyls,” the latter of which refers to alkenyl moieties having substituents replacing a hydrogen on one or more carbons of the hydrocarbon backbone. Such substituents can include, for example, alkyl, alkenyl, alkynyl, halogens, hydroxyl, aryl alkylcarbonyloxy, arylcarbonyloxy, alkoxycarbonyl oxy, aryloxycarbonyloxy, —COOH, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “alkynyl” includes unsaturated aliphatic groups analogous in length and possible substitution to the alkyls described above, but which contain at least one triple bond. For example, the term “alkynyl” includes straight-chain alkynyl groups (e.g., ethynyl, propynyl, butynyl, pentynyl, hexynyl, heptynyl, octynyl, nonynyl, decynyl, etc.), branched-chain alkynyl groups, and cycloalkyl or cycloalkenyl substituted alkynyl groups. The term “alkynyl” further includes alkynyl groups which include oxygen, nitrogen, sulfur or phosphorous atoms replacing one or more carbons of the hydrocarbon backbone. In certain embodiments, a straight chain or branched chain alkynyl group has 6 or fewer carbon atoms in its backbone (e.g., C₂-C₆ for straight chain, C₃-C₆ for branched chain). The term C₂-C₆ includes alkynyl groups containing 2 to 6 carbon atoms.

Moreover, the term “alkynyl” includes both “unsubstituted alkynyls” and “substituted alkynyls,” the latter of which refers to alkynyl moieties having substituents replacing a hydrogen on one or more carbons of the hydrocarbon backbone. Such substituents can include, for example, alkyl, alkenyl, alkynyl, halogens, hydroxyl, aryl, alkylcarbonyloxy, arylcarbonyloxy, alkoxycarbonyloxy, aryloxycarbonyloxy, —COOH, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “acyl” includes compounds and moieties which contain the acyl radical (CH₃CO—). It also includes substituted acyl moieties. The term “substituted acyl” includes acyl groups where one or more of the hydrogen atoms are replaced by for example, alkyl, alkenyl, alkynyl, halogens, hydroxyl, aryl, alkylcarbonyloxy, arylcarbonyloxy, alkoxycarbonyloxy, aryloxycarbonyloxy, —COOH, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “acylamino” includes moieties wherein: an acyl moiety is bonded to an amino group. For example, the term includes alkylcarbonylamino, arylcarbonylamino, carbamoyl and ureido groups.

The terms “alkoxyalkyl,” “alkylaminoalkyl” and “thioalkoxyalkyl” include alkyl groups, as described above, which further include oxygen, nitrogen or sulfur atoms replacing one or more carbons of the hydrocarbon backbone, e.g., oxygen, nitrogen or sulfur atoms.

The term “alkoxy” includes substituted and unsubstituted alkyl, alkenyl, and alkynyl groups covalently linked to an oxygen atom. Examples of alkoxy groups include methoxy, ethoxy, isopropyloxy, propoxy, butoxy, and pentoxy groups.

Examples of substituted alkoxy groups include halogenated alkoxy groups. The alkoxy groups can be substituted with groups such as alkyl, alkenyl, alkynyl, halogen, hydroxyl, aryl, alkylcarbonyloxy, arylcarbonyloxy, alkoxycarbonyloxy, aryloxycarbonyloxy, —COOH, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy, trichloromethoxy, etc.

The term “amine” or “amino” includes compounds where a nitrogen atom is covalently bonded to at least one carbon or heteroatom. The term includes “alkyl amino” which comprises groups and compounds wherein: the nitrogen is bound to at least one additional alkyl group. The term “dialkyl amino” includes groups wherein: the nitrogen atom is bound to at least two additional alkyl groups. The term “arylamino” and “diarylamino” include groups in which the nitrogen is bound to at least one or two aryl groups, respectively. The term “alkylarylamino,” “alkylaminoaryl” or “arylaminoalkyl” refers to an amino group which is bound to at least one alkyl group and at least one aryl group. The term “alkaminoalkyl” refers to an alkyl, alkenyl, or alkynyl group bound to a nitrogen atom which is also bound to an alkyl group.

The term “amide,” “amido” or “aminocarbonyl” includes compounds or moieties which contain a nitrogen atom which is bound to the carbon of a carbonyl or a thiocarbonyl group. The term includes “alkaminocarbonyl” or “alkylaminocarbonyl” groups which include alkyl, alkenyl, aryl or alkynyl groups bound to an amino group bound to a carbonyl group. It includes arylaminocarbonyl and arylcarbonylamino groups, which include aryl or heteroaryl moieties bound to an amino group that is bound to the carbon of a carbonyl or thiocarbonyl group. The terms “alkylaminocarbonyl,” “alkenylaminocarbonyl,” “alkynylaminocarbonyl,” “arylaminocarbonyl,” “alkylcarbonylamino,” “alkenyl carbonylamino,” “alkynylcarbonylamino,” and “arylcarbonylamino” are included in term “amide.” Amides also include urea groups (aminocarbonylamino) and carbamates (oxycarbonylamino).

The term “carbonyl” or “carboxy” includes compounds and moieties which contain a carbon connected with a double bond to an oxygen atom. The carbonyl can be further substituted with any moiety which allows the compounds of the invention to perform its intended function. For example, carbonyl moieties may be substituted with alkyls, alkenyls, alkynyls, aryls, alkoxy, aminos, etc. Examples of moieties which contain a carbonyl include aldehydes, ketones, carboxylic acids, amides, esters, anhydrides, etc. The term “carboxy” further includes the structure of —COOH and —COO⁻.

The term “oximyl” includes compounds and moieties that contain a carbon connected with a double bond to a nitrogen atom, which is, in turn connected to a hydroxyl or an alkoxyl group. The term “hydrazinyl” includes compounds and moieties that contain a carbon connected with a double bond to a nitrogen atom, which is, in turn, connected to an amino group.

The term “thiocarbonyl” or “thiocarboxy” includes compounds and moieties which contain a carbon connected with a double bond to a sulfur atom.

The term “ether” includes compounds or moieties which contain an oxygen bonded to two different carbon atoms or heteroatoms. For example, the term includes “alkoxyalkyl” which refers to an alkyl, alkenyl, or alkynyl group covalently bonded to an oxygen atom which is covalently bonded to another alkyl group.

The term “thioether” includes compounds and moieties which contain a sulfur atom bonded to two different carbon or hetero atoms. Examples of thioethers include, but are not limited to, alkthioalkyls, alkthioalkenyls, and alkthioalkynyls. The term “alkthioalkyls” include compounds with an alkyl, alkenyl, or alkynyl group bonded to a sulfur atom which is bonded to an alkyl group. Similarly, the term “alkthioalkenyls” and “alkthioalkynyl” refer to compounds or moieties in which an alkyl, alkenyl or alkynyl group is bonded to a sulfur atom that is covalently bonded to an alkenyl or alkynyl group, respectively.

The term “sulfonyl” includes moieties containing a sulfonyl functional group (e.g., SO₂) attached to two carbons via a covalent bond to the sulfur atom of the sulfonyl functional group.

The term “hydroxyl” or “hydroxyl” includes groups with an —OH or —O⁻.

The term “halogen” includes fluorine, bromine, chlorine, iodine, etc.

The term “heteroatom” includes atoms of any element other than carbon or hydrogen. Preferred heteroatoms are nitrogen, oxygen, sulfur and phosphorus.

The invention provides compositions which include a therapeutically-effective amount or dose of a transcription factor modulating compound and/or a compound identified in any of the instant assays and one or more carriers (e.g., pharmaceutically acceptable additives and/or diluents). The pharmaceutical compositions of the invention may comprise any compound described in this application as a transcription factor modulating compound, an AraC family polypeptide modulating compound, a MarA family polypeptide modulating compound, a MarA family inhibiting compound, an AraC family inhibiting compound or a compound of formula I, II, III, IV, IVa, VI, VIa, VII, or VIII or a compound of Table 2. Each of these compounds may be used alone or in combination as a part of a pharmaceutical composition of the invention.

The invention pertains to pharmaceutical compositions comprising an effective amount of a transcription factor modulating compound (e.g., a MarA family polypeptide modulating compound or an AraC family polypeptide modulating compound), and a pharmaceutically acceptable carrier. In one embodiment, the transcription factor modulating compound is of formula I, II, III, IV, IVa, VI, VIa, VII, or VIII or a compound of Table 2.

In one embodiment, the present invention provides a pharmaceutical composition comprising a pharmaceutically acceptable carrier and a transcription factor modulating compound, wherein: said compound is of formula I, II, III, IV, IVa, VI, VIa, VII, or VIII or a compound of Table 2. In another embodiment, the pharmaceutical composition can further comprise an antibiotic.

In one embodiment, the transcription factor modulating compound (e.g., a compound of formula I, II, III, IV, IVa, VI, VIa, VII, or VIII or a compound of Table 2) is administered in combination with an antibiotic. The language “in combination with” an antibiotic includes co-administration of the transcription factor modulating compound and with an antibiotic, administration of the transcription factor modulating compound first, followed by administration of an antibiotic, and administration of the antibiotic first, followed by administration of the transcription factor modulating compound. The transcription factor modulating compound can be administered substantially at the same time as the antibiotic or at substantially different times as the antibiotic. Optimal administration rates for a given protocol of administration of the transcription factor modulating and/or the antibiotic can be readily ascertained by those skilled in the art using conventional dosage determination tests conducted with regard to the specific compounds being utilized, the particular compositions formulated, the mode of application, the particular site of administration and the like.

The term “antibiotic” refers to chemotherapeutic agents that inhibit or abolish the growth of microbial cells (e.g., bacteria or fungi). Suitable antibiotics include, but are not limited to, aminoglycosides, ancimycins, carbacephams, cephalosporins, glycopeptides, macrolides, monobactems, penicillins, polypeptides, quinolines, sulphonamides, tetracyclines and the like. One of skill in the art using conventional medical diagnoses would be able to determine the appropriate antibiotic agent to administer in combination with the transcription factor modulating compounds of the invention.

The language “effective amount” of the compound is that amount necessary or sufficient to treat, prevent or ameliorate a bacterial infection (e.g., pneumonia, urinary tract infection, kidney infection), biofilm formation, bacterial growth (e.g., on a contact lens or on a medical indwelling device), corneal ulcers and burn wounds in a subject. The effective amount can vary depending on such factors as the size and weight of the subject, the type of illness, etc. One of ordinary skill in the art would be able to study the aforementioned factors and make the determination regarding the effective amount of the transcription factor modulating compounds without undue experimentation.

The term “subject” includes plants and animals (e.g., vertebrates, amphibians, fish, mammals, e.g., cats, dogs, horses, pigs, cows, sheep, rodents, rabbits, squirrels, bears, primates (e.g., chimpanzees, gorillas, and humans) which are capable of suffering from a bacterial associated disorder. The term “subject” also comprises immunocompromised subjects, who may be at a higher risk for infection.

The terms “preventing” and “prevention” include the administration of an effective amount of the transcription factor modulating compound to prevent a bacterial infection (e.g., pneumonia, urinary tract infection, kidney infection), biofilm formation, bacterial growth (e.g., on a contact lens or a medical indwelling device) from occurring.

The terms “treating” and “treatment” include the administration to a subject an effective amount of the transcription factor modulating compound to treat the subject for a bacterial infection (e.g., pneumonia, urinary tract infection, kidney infection), biofilm formation, bacterial growth (e.g., on a contact lens or on a medical indwelling device), corneal ulcers and burn wounds.

The transcription factor modulating compounds of the invention (e.g., a compound of formula I, II, III, IV, IVa, VI, VIa, VII, or VIII or a compound of Table 2) that are basic in nature are capable of forming a wide variety of salts with various inorganic and organic acids. The acids that may be used to prepare pharmaceutically acceptable acid addition salts of the transcription factor modulating compounds of the invention that are basic in nature are those that form non-toxic acid addition salts, i.e., salts containing pharmaceutically acceptable anions, such as the hydrochloride, hydrobromide, hydroiodide, nitrate, sulfate, bisulfate, phosphate, acid phosphate, isonicotinate, acetate, lactate, salicylate, citrate, acid citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and palmoate (i.e., 1,1′-methylene-bis-(2-hydroxy-3-naphthoate)] salts. Although such salts must be pharmaceutically acceptable for administration to a subject, e.g., a mammal, it is often desirable in practice to initially isolate a transcription factor modulating compound from the reaction mixture as a pharmaceutically unacceptable salt and then simply convert the latter back to the free base compound by treatment with an alkaline reagent and subsequently convert the latter free base to a pharmaceutically acceptable acid addition salt. The acid addition salts of the base compounds of this invention are readily prepared by treating the base compound with a substantially equivalent amount of the chosen mineral or organic acid in an aqueous solvent medium or in a suitable organic solvent, such as methanol or ethanol. Upon careful evaporation of the solvent, the desired solid salt is readily obtained. The preparation of other transcription factor modulating compounds not specifically described in the experimental section can be accomplished using combinations of the described reactions that will be apparent to those skilled in the art.

The transcription factor modulating compounds of the invention (e.g., a compound of formula I, II, III, IV, IVa, VI, VIa, VII, or VIII or a compound of Table 2) that are acidic in nature are capable of forming a wide variety of base salts. The chemical bases that may be used as reagents to prepare pharmaceutically acceptable base salts of those transcription factor modulating compounds that are acidic in nature are those that form non-toxic base salts with such compounds. Such non-toxic base salts include, but are not limited to those derived from such pharmaceutically acceptable cations such as alkali metal cations (e.g., potassium and sodium) and alkaline earth metal cations (e.g., calcium and magnesium), ammonium or water-soluble amine addition salts such as N-methylglucamine-(meglumine), and the lower alkanolamnionium and other base salts of pharmaceutically acceptable organic amines. The pharmaceutically acceptable base addition salts of transcription factor modulating compounds that are acidic in nature may be formed with pharmaceutically acceptable cations by conventional methods. Thus, these salts may be readily prepared by treating the transcription factor modulating compounds of the invention with an aqueous solution of the desired pharmaceutically acceptable cation and evaporating the resulting solution to dryness, preferably under reduced pressure. Alternatively, a lower alkyl alcohol solution of the transcription factor modulating compounds of the invention may be mixed with an alkoxide of the desired metal and the solution subsequently evaporated to dryness.

These compositions may also contain adjuvants such as preservatives, wetting agents, emulsifying agents and dispersing agents. Prevention of the action of microbes may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin. In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Pharmaceutical compositions of the present invention may be administered to epithelial surfaces of the body orally, parenterally, topically, rectally, nasally, intravaginally, intracisternally. They are, of course, given by forms suitable for each administration route. For example, they are administered in tablets or capsule form, by injection, inhalation, eye lotion, ointment, etc., administration by injection, infusion or inhalation; topical by lotion or ointment; and rectal or vaginal suppositories.

The phrases “parenteral administration” and “administered parenterally” as used herein include modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally,” as used herein, includes the administration of the transcription factor modulating compound of the invention other than directly into the central nervous system, such that it enters the subject's system and, thus, is subject to metabolism and other like processes, for example, subcutaneous administration.

In some methods, the compositions of the invention can be topically administered to any epithelial surface. An “epithelial surface” include an area of tissue that covers external surfaces of a body, or which lines hollow structures including, but not limited to, cutaneous and mucosal surfaces. Such epithelial surfaces include oral, pharyngeal, esophageal, pulmonary, ocular, aural, nasal, buccal, lingual, vaginal, cervical, genitourinary, alimentary, and anorectal surfaces.

Compositions can be formulated in a variety of conventional forms employed for topical administration. These include, for example, semi-solid and liquid dosage forms, such as liquid solutions or suspensions, suppositories, douches, enemas, gels, creams, emulsions, lotions, slurries, powders, sprays, lipsticks, foams, pastes, toothpastes, ointments, salves, balms, douches, drops, troches, chewing gums, lozenges, mouthwashes, rinses.

Conventionally used carriers for topical applications include pectin, gelatin and derivatives thereof, polylactic acid or polyglycolic acid polymers or copolymers thereof, cellulose derivatives such as methyl cellulose, carboxymethyl cellulose, or oxidized cellulose, guar gum, acacia gum, karaya gum, tragacanth gum, bentonite, agar, carbomer, bladderwrack, ceratonia, dextran and derivatives thereof, ghatti gum, hectorite, ispaghula husk, polyvinypyrrolidone, silica and derivatives thereof, xanthan gum, kaolin, talc, starch and derivatives thereof, paraffin, water, vegetable and animal oils, polyethylene, polyethylene oxide, polyethylene glycol, polypropylene glycol, glycerol, ethanol, propanol, propylene glycol (glycols, alcohols), fixed oils, sodium, potassium, aluminum, magnesium or calcium salts (such as chloride, carbonate, bicarbonate, citrate, gluconate, lactate, acetate, gluceptate or tartrate).

Standard composition strategies for topical agents can be applied to the transcription factor modulating compounds of the invention or a pharmaceutically acceptable salt thereof in order to enhance the persistence and residence time of the drug, and to improve the prophylactic efficacy achieved.

For topical application to be used in the lower intestinal tract or vaginally, a rectal suppository, a suitable enema, a gel, an ointment, a solution, a suspension or an insert can be used. Topical transdermal patches may also be used. Transdermal patches have the added advantage of providing controlled delivery of the compositions of the invention to the body. Such dosage forms can be made by dissolving or dispersing the agent in the proper medium.

Compositions of the invention can be administered in the form of suppositories for rectal or vaginal administration. These can be prepared by mixing the agent with a suitable non-irritating carrier which is solid at room temperature but liquid at rectal temperature and therefore will melt in the rectum or vagina to release the drug. Such materials include cocoa butter, beeswax, polyethylene glycols, a suppository wax or a salicylate that is solid at room temperature, but liquid at body temperature and, therefore, will melt in the rectum or vaginal cavity and release the active agent. Compositions which are suitable for vaginal administration also include pessaries, tampons, creams, gels, pastes, foams, films, or spray compositions containing such carriers as are known in the art to be appropriate. The carrier employed in the pharmaceutical compositions of the invention should be compatible with vaginal administration.

For ophthalmic applications, the pharmaceutical compositions can be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the compositions can be formulated in an ointment such as petrolatum. Exemplary ophthalmic compositions include eye ointments, powders, solutions and the like.

Powders and sprays can contain, in addition to the transcription factor modulating compound of the invention, carriers such as lactose, talc, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Ordinarily, an aqueous aerosol is made by formulating an aqueous solution or suspension of the transcription factor modulating compound (e.g., a compound of formula I, II, III, IV, IVa, VI, VIa, VII, or VIII or a compound of Table 2) together with conventional pharmaceutically acceptable carriers and stabilizers. The carriers and stabilizers vary with the requirements of the particular compound, but typically include nonionic surfactants (e.g., Tweens, Pluronics, polyethylene glycol and the like), proteins like serum albumin, sorbitan esters, oleic acid, lecithin, amino acids such as glycine, buffers, salts, sugars or sugar alcohols. Aerosols generally are prepared from isotonic solutions. Generation of the aerosol or any other means of delivery of the present invention may be accomplished by any of the methods known in the art. For example, in the case of aerosol delivery, the compound is supplied in a finely divided form along with any suitable carrier with a propellant.

Liquefied propellants are typically gases at ambient conditions and are condensed under pressure. The propellant may be any acceptable and known in the art including propane and butane, or other lower alkanes, such as those of up to 5 carbons. The composition is held within a container with an appropriate propellant and valve, and maintained at elevated pressure until released by action of the valve.

Compositions of the invention can also be orally administered in any orally-acceptable dosage form including, but not limited to, capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of sucrose octasulfate and/or antibiotic or contraceptive agent(s) as an active ingredient. A transcription factor modulating compound may also be administered as a bolus, electuary or paste. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions are required for oral use, the active ingredient is combined with emulsifying and suspending agents. If desired, certain sweetening, flavoring or coloring agents may also be added. Tablets, and other solid dosage forms, such as dragees, capsules, pills and granules, may be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the transcription factor modulating compound only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients. Liquid dosage forms for oral administration include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the transcription factor modulating compounds of the invention, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Sterile injectable forms of the compositions of this invention can be aqueous or oleaginous suspension. These suspensions may be formulated according to techniques known in the art using suitable dispersing or wetting agents and suspending agents.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions. The sterile injectable preparation may also be a sterile injectable solution or suspension in a nontoxic parenterally-acceptable diluent or solvent, for example, as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant. The transcription factor modulating compound or a pharmaceutically acceptable salt thereof will represent some percentage of the total dose in other dosage forms in a material forming a combination product, including liquid solutions or suspensions, suppositories, douches, enemas, gels, creams, emulsions, lotions slurries, soaps, shampoos, detergents, powders, sprays, lipsticks, foams, pastes, toothpastes, ointments, salves, balms, douches, drops, troches, lozenges, mouthwashes, rinses and others.

In one embodiment, the transcription factor modulating compounds of the invention may be administered prophylactically. For prophylactic applications, the pharmaceutical composition of the invention can be applied prior to potential infection. The timing of application prior to potential infection can be optimized to maximize the prophylactic effectiveness of the compound. The timing of application will vary depending on the mode of administration, doses, the stability and effectiveness of composition, the frequency of the dosage, e.g., single application or multiple dosage. One skilled in the art will be able to determine the most appropriate time interval required to maximize prophylactic effectiveness of the compound.

A transcription factor modulating compound, e.g., a compound of formula I, II, III, IV, IVa, VI, VIa, VII, or VIII or a compound of Table 2, when present in a composition will generally be present in an amount from about 0.000001% to about 100%, more preferably from about 0.001% to about 50%, and most preferably from about 0.01% to about 25% of total weight.

For compositions of the present invention comprising a carrier, the composition comprises, for example, from about 1% to about 99%, preferably from about 50% to about 99%, and most preferably from about 75% to about 99% by weight of at least one carrier.

Also, the separate components of the compositions of the invention may be preblended or each component may be added separately to the same environment according to a predetermined dosage for the purpose of achieving the desired concentration level of the treatment components and so long as the components eventually come into intimate admixture with each other. Further, the present invention may be administered or delivered on a continuous or intermittent basis.

The transcription factor modulating compounds may be formulated in a composition suitable for use in environments including industry, pharmaceutics, household, and personal care. For example, compounds of the present invention are also useful as active antimicrobial ingredients in household products such as cleansers, detergents, disinfectants, dishwashing liquids, soaps and detergents. In an embodiment, the transcription factor modulating compounds of the present invention may be delivered in an amount and form effective for the prevention of colonization, removal or death of microbes. The compositions of the invention for household use comprise, for example, at least one transcription factor modulating compound of the invention and at least one suitable carrier. For example, the composition may comprise from about 0.00001% to about 50%, preferably from about 0.0001% to about 25%, for example, from about 0.0005% to about 10% by weight of the modulating compound based on the weight percentage of the total composition.

The transcription factor modulating compounds may also be used in hygiene compositions for personal care. For instance, compounds of the invention can be used as an active ingredient in personal care products such as facial cleansers, astringents, body wash, shampoos, conditioners, cosmetics and other hygiene products. The hygiene composition may comprise any carrier or vehicle known in the art to obtain the desired form (such as solid, liquid, semisolid or aerosol) as long as the effects of the compound of the present invention are not impaired. Methods of preparation of hygiene compositions are not described herein in detail, but are known in the art. For discussion of such methods, see The CTFA Cosmetic Ingredient Handbook, Second Edition, 1992, and pages 5-484 of A Formulary of Cosmetic Preparations (Vol. 2, Chapters 7-16), incorporated herein by reference.

A dentifrice or mouthwash containing the compounds of the invention may be formulated by adding the compounds of the invention to dentifrice and mouthwash formulations, e.g., as set forth in Remington's Pharmaceutical Sciences, 18th Ed., Mack Publishing Co., 1990, Chapter 109 (incorporated herein by reference in its entirety). The dentifrice may be formulated as a gel, paste, powder or slurry. The dentifrice may include binders, abrasives, flavoring agents, foaming agents and humectants. Mouthwash formulations are known in the art, and the compounds of the invention may be advantageously added to them.

The hygiene composition for use in personal care comprise generally at least one transcription factor modulating compound of the present application and at least one suitable carrier. For example, the composition may comprise from about 0.00001% to about 50%, preferably from about 0.0001% to about 25%, for example, from about 0.0005% to about 10% by weight of the transcription factor modulating compound of the invention based on the weight percentage of the total composition.

The composition can be formulated as a cleaning product, e.g., a household or an industrial cleaner to remove, prevent, inhibit, or modulate biofilm development. These compositions may include compounds such as disinfectants, soaps, detergents, as well as other surfactants. Examples of surfactants include, for example, sodium dodecyl sulfate; quaternary ammonium compounds; alkyl pyridinium iodides; TWEEN 80, TWEEN 85, TRITON X-100; BRIJ 56; biological surfactants, rhamnolipid, surfactin, visconsin and sulfonates. The compositions of the invention may be applied in known areas and surfaces where disinfection is required, including, but not limited to, drains, shower curtains, grout, toilets and flooring. A particular application is on hospital surfaces and medical instruments. The disinfectant of the invention may be useful as a disinfectant for bacteria such as, but not limited to, Pseudomonadaceae, Azatobacteraceae, Rhizabiceae, Mthylococcaceae, Halobacteriaceae, Acetobacteraceae, Legionellaceae, Neisseriaceae and other genera.

The transcription factor modulating compounds of the present invention may be used in industry. In the industrial setting, the presence of microbes can be problematic, as microbes are often responsible for industrial contamination and biofouling. Compositions of the invention for industrial applications may comprise an effective amount of a compound in a composition for industrial use with at least one acceptable carrier or vehicle known in the art to be useful in the treatment of such systems. Such carriers or vehicles may include diluents, deflocculating agents, penetrants, spreading agents, surfactants, suspending agents, wetting agents, stabilizing agents, compatibility agents, sticking agents, waxes, oils, co-solvents, coupling agents, foams, antifoaming agents, natural or synthetic polymers, elastomers and synergists. Methods of preparation, delivery systems and carriers for such compositions are not described here in detail, but are known in the art. For its discussion of such methods, U.S. Pat. No. 5,939,086 is herein incorporated by reference. Furthermore, the preferred amount of the composition to be used may vary according to the active ingredient(s) and situation in which the composition is being applied.

The transcription factor modulating compounds, e.g., compounds of formula I, II, III, IV, IVa, VI, VIa, VII, or VIII or a compound of Table 2, may be useful in nonaqueous environments. Such nonaqueous environments may include, but are not limited to, terrestrial environments, dry surfaces or semi-dry surfaces in which the compound or composition is applied in a manner and amount suitable for the situation. The transcription factor modulating compounds, e.g., compounds of formula I, II, III, IV, IVa, VI, VIa, VII, or VIII or a compound of Table 2, may be used to form coatings or layers on a variety of substrates including personal care products (such as toothbrushes, contact lens cases and dental equipment), healthcare products, household products, food preparation surfaces and packaging, and laboratory and scientific equipment. Further, other substrates include medical devices such as catheters, urological devices, blood collection and transfer devices, tracheotomy devices, intraocular lenses, wound dressings, sutures, surgical staples, membranes, shunts, gloves, tissue patches, prosthetic devices (e.g., heart valves) and wound drainage tubes. Other substrates include textile products such as carpets and fabrics, paints and joint cement. A further use is as an antimicrobial soil fumigant.

The present invention also provides a process for the production of an antibiofouling composition for industrial use. Such process comprises bringing at least one of any industrially acceptable carrier known in the art into intimate admixture with a transcription factor modulating compound of the present invention. The carrier may be any suitable carrier discussed above or known in the art.

The suitable antibiofouling compositions may be in any acceptable form for delivery of the composition to a site potentially having, or having at least one living microbe. The antibiofouling compositions may be delivered with at least one suitably selected carrier as hereinbefore discussed using standard formulations. The mode of delivery may be such as to have a binding inhibiting effective amount of the antibiofouling composition at a site potentially having, or having at least one living microbe. The antibiofouling compositions of the present invention are useful in treating microbial growth that contributes to biofouling, such as scum or slime formation, in these aqueous environments. Examples of industrial processes in which these compounds might be effective include cooling water systems, reverse osmosis membranes, pulp and paper systems, air washer systems and the food processing industry. The antibiofouling composition may be delivered in an amount and form effective for the prevention, removal or termination of microbes. The antibiofouling composition of the present invention generally comprise at least one compound of the invention. The composition may comprise from about 0.001% to about 50%, about 0.003% to about 15%, about 0.01% to about 5% by weight of the compound of the invention based on the weight percentage of the total composition. The amount of antibiofouling composition may be delivered in an amount of about 1 mg/l to about 1000 mg/l, from about 2 mg/l to about 500 mg/l, or from about 20 mg/l to about 140 mg/l.

Antibiofouling compositions for water treatment generally comprise transcription factor modulating compounds of the invention in amounts from about 0.001% to about 50% by weight of the total composition. Other components in the antibiofouling compositions (used at 0.1% to 50%) may include, for example, 2-bromo-2-nitropropane-1,3-diol (BNPD), β-nitrostyrene (BNS), dodecylguanidine hydrochloride, 2,2-dibromo-3-nitrilopropionamide (DBNPA), glutaraldehyde, isothiazolin, methylene bis(thiocyanate), triazines, n-alkyl dimethylbenzylammonium chloride, trisodium phosphate-based, antimicrobials, tributyltin oxide, oxazolidines, tetrakis(hydroxymethyl)phosphonium sulfate (THPS), phenols, chromated copper arsenate, zinc or copper pyrithione, carbamates, sodium or calcium hypochlorite, sodium bromide, halohydantoins (Br, Cl), or mixtures thereof.

Other possible components in the compositions of the invention include biodispersants (about 0.1% to about 15% by weight of the total composition), water, glycols (about 20-30%) or Pluronic (at approximately 7% by weight of the total composition). The concentration of antibiofouling composition for continuous or semi-continuous use is about 5 to about 70 mg/l.

Antibiofouling compositions for industrial water treatment may comprise compounds of the invention in amounts from about 0.001% to about 50% based on the weight of the total composition. The amount of compound of the invention in antibiofouling compositions for aqueous water treatment may be adjusted depending on the particular environment. Shock dose ranges are generally about 20 to about 140 mg/l; the concentration for semi-continuous use is about 0.5 times of these concentrations.

The invention also pertains, at least in part, to a method of regulating biofilm development. The method includes administering a composition which contains a transcription factor modulating compound of the invention. The composition can also include other components which enhance the ability of the composition to degrade biofilms.

The transcription factor modulating compounds may also be incorporated into polymers, such as polysaccharides (cellulose, cellulose derivatives, starch, pectins, alginate, chitin, guar, carrageenan), glycol polymers, polyesters, polyurethanes, polyacrylates, polyacrylonitrile, polyamides (e.g., nylons), polyolefins, polystyrenes, vinyl polymers, polypropylene, silks or biopolymers. The transcription factor modulating compounds may be conjugated to any polymeric material such as those with the following specified functionality: 1) carboxy acid, 2) amino group, 3) hydroxyl group and/or 4) haloalkyl group. The composition for treatment of nonaqueous environments may be comprise at least one transcription factor modulating compound (e.g., a compound of formula I, II, III, IV, IVa, VI, VIa, VII, or VIII or a compound of Table 2) and at least one suitable carrier. In an embodiment, the composition comprises from about 0.001% to about 75%, about 0.01% to about 50%, and about 0.1% to about 25% by weight of a transcription factor modulating compound of the invention based on the weight percentage of the total composition.

The transcription factor modulating compounds and compositions may also be useful in aqueous environments. “Aqueous environments” include any type of system containing water, including, but not limited to, natural bodies of water such as lakes or ponds; artificial, recreational bodies of water such as swimming pools and hot tubs; and drinking reservoirs, such as wells. The compositions of the present invention may be useful in treating microbial growth in these aqueous environments and may be applied, for example, at or near the surface of water.

The compositions of the invention for treatment of aqueous environments may comprise at least one transcription factor modulating compound and at least one suitable carrier. In an embodiment, the composition comprises from about 0.001% to about 50%, about 0.003% to about 15%, about 0.01% to about 5% by weight of the compound of the invention based on the weight percentage of the total composition.

The practice of the present invention will employ, unless otherwise indicated, conventional techniques of cell biology, cell culture, molecular biology, genetics, microbiology, recombinant DNA, and immunology, which are within the skill of the art. Such techniques are explained fully in the literature. See, for example, Genetics; Molecular Cloning A Laboratory Manual, 2nd Ed., ed. by Sambrook, J. et al. (Cold Spring Harbor Laboratory Press (1989)); Short Protocols in Molecular Biology, 3rd Ed., ed. by F. Ausubel et al. (Wiley, NY (1995)); DNA Cloning, Volumes I and II (D. N. Glover ed., 1985); Oligonucleotide Synthesis (M. J. Gait ed. (1984)); Mullis et al. U.S. Pat. No. 4,683,195; Nucleic Acid Hybridization (B. D. Hames & S. J. Higgins eds. (1984)); the treatise, Methods in Enzymology (Academic Press, Inc., N.Y); Immunochemical Methods in Cell And Molecular Biology (Mayer and Walker, eds., Academic Press, London (1987)); Handbook of Experimental Immunology, Volumes I-IV (D. M. Weir and C. C. Blackwell, eds. (1986)); and J. Miller, Experiments in Molecular Genetics (Cold Spring Harbor Press, Cold Spring Harbor, N.Y. (1972)).

The contents of all references, patent applications and patents, cited throughout this application are hereby expressly incorporated by reference. Each reference disclosed herein is incorporated by reference herein in its entirety. Any patent application to which this application claims priority is also incorporated by reference herein in its entirety.

### EXEMPLIFICATION OF THE INVENTION

### Example 1

### Synthesis of Selected Compounds of the Invention

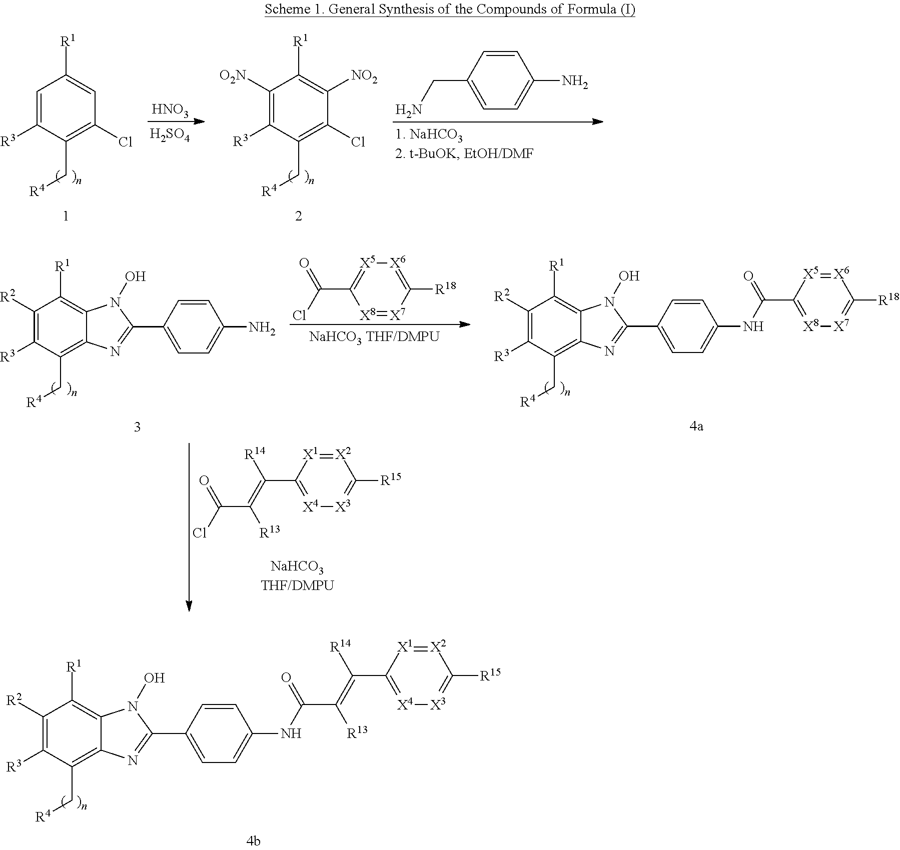

### General Synthesis of Intermediate 3

To a solution of a 2-chlorophenylacetic acid (4.25 g, 25.0 mmol) (1) in concentrated H₂SO₄ (15.0 mL) at room temperature was added a solution of fuming HNO₃ (3.8 mL) in concentrated H₂SO₄ (7.5 mL) in portions. After 1 hour, the solution was heated to 60° C. for another 4 hours. After cooling to room temperature, the mixture was poured into stirring ice water to precipitate the product. The product 2 was collected on fritted funnel rinsing with water. The desired product was further dried under high vacuum to afford 4.88 g in 75% yield as a white solid. To a solution of 4-aminobenzyl amine (3.5 mL, 31.3 mmol) and powdered NaHCO₃ (15.8 g, 188 mmol) in anhydrous DMF (50 mL) at room temperature was added a solution of 2-chloro-3,5-dinitrophenyl acetic acid (2) (4.27 g, 25.0 mmol) in anhydrous DMF (5.0 mL) dropwise via addition funnel over a 1 hour period. After another 4 hours or determined complete by HPLC, the solution was diluted with anhydrous absolute ethanol (100 mL) then powdered potassium tert-butoxide (14.0 g, 125 mmol) was added in portions. The solution was heated to 60° C. for 6 hours. After cooling to room temperature, the solution was poured into stirring solution of water (0.4 L), then adjusted to a pH=6 with 1M HCl. The slowly stirring suspension was cooled with an ice bath to facilitate solidification. The suspended product 3 was collected on a fine fritted funnel rinsing with water until the eluent was colorless. The orange solid 3 was further dried under high vacuum.

### General Synthesis of Compounds 4a and 4b

To a solution of intermediate 3 (1.00 mmol) in anhydrous pyridine (2.0 mL) was added the appropriate acid chloride (2.50 mmol) at room temperature. After stiffing for 2-3 hours, the solution was diluted with 3M NaOH (6.0 mL) and stirred for another hour. The deep amber solution was transferred to an Erlenmeyer flask or beaker through dilution with water (100 mL) and then acidified with saturated citric acid. The resulting precipitate was collected on a sintered funnel rinsing with water. The crude product (4a or 4b) was further purified either by preparatory HPLC, or by recrystallization in hot ethanol or a mixture of hot ethanol and chloroform. Compounds J, Q, R, V, AA and AH were synthesized by this matter.

### Compound J: (2-{4-[(E)-3-(4-Acetyl-phenyl)-acryloylamino]-phenyl}-1-hydroxy-6-nitro-1H-benzoimidazol-4-yl)-acetic acid

¹H NMR (300 MHz, DMSO-d₆, a drop of 3M NaOH in D₂O): δ 8.51 (d, J=8.7 Hz, 2H), 8.14 (d, J=2.4 Hz, 1H), 7.92 (d, J=8.4 Hz, 2H), 7.80 (d, J=2.4 Hz, 1H), 7.66 (d, J=8.4 Hz, 2H), 7.54 (d, J=8.7 Hz, 2H), 7.24 (d, J=15.6 Hz, 1H), 6.94 (d, J=15.9 Hz, 1H), 3.66 (s, 2H), 3.19 (s, 3H). MS (ESI, positive): calcd, 500.47; found, [M+1]⁺=501.25.

### Compound R: {2-[4-(4-Acetyl-benzoylamino)-phenyl]-1-hydroxy-6-nitro-1H-benzoimidazol-4-yl}-acetic acid

¹H NMR (300 MHz, DMSO-d₆): δ 10.85 (s, 1H), 8.36 (d, J=8.7 Hz, 2H), 8.27 (d, J=2.1 Hz, 1H), 8.16-8.04 (m, 7H), 4.10 (s, 2H), 2.65 (s, 3H). MS (ESI, positive): calcd, 474.43; found, [M+1]⁺=475.45.

### Compound V: (2-{4-[(E)-3-(6-Acetyl-pyridin-3-yl)-acryloylamino]-phenyl}-1-hydroxy-6-nitro-1H-benzoimidazol-4-yl)-acetic acid

¹H NMR (300 MHz, DMSO-d₆): δ 10.61 (s, 1H), 8.94 (d, J=1.8 Hz, 1H), 8.29 (d, J=8.7 Hz, 2H), 8.20 (dd, J=2.1, 8.4 Hz, 1H), 8.06-7.92 (m, 3H), 7.80 (d, J=8.7 Hz, 2H), 7.72 (d, J=15.6 Hz, 1H), 7.07 (d, J=15.9 Hz, 1H), 3.91 (s, 2H), 2.60 (s, 3H). MS (ESI, positive): calcd, 501.45; found, [M+1]⁺=502.25.

### Compound AA: {1-Hydroxy-2-[4-(4-imidazol-1-yl-benzoylamino)-phenyl]-6-nitro-1H-benzoimidazol-4-yl}-acetic acid

¹H NMR (300 MHz, DMSO-d₆): δ 10.73 (s, 1H), 9.67 (s, 1H), 8.38-8.35 (m, 3H), 8.28-8.24 (m, 3H), 8.11-7.99 (m, 5H), 7.89 (s, 1H), 4.10 (s, 2H). MS (ESI, positive): calcd, 498.45; found, [M+1]⁺=499.25.

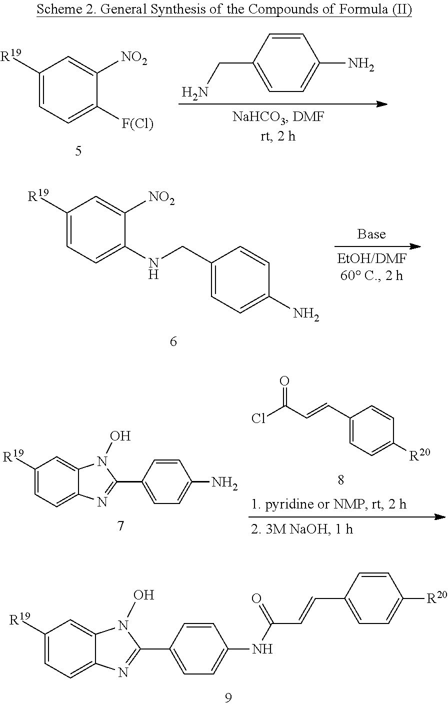

### Preparation of Intermediate 6

To a solution of 4-aminobenzyl amine (225 mmol) and powdered NaHCO₃ (1125 mmol) in anhydrous DMF (300 mL) at was added a substituted 2-nitrofluoro or 2-nitrochloro dibenzene (5) (150 mmol) dropwise at room temperature. After 2 hours, the solution was slowly diluted with water (1000 mL) to precipitate the product 6, which was collected on a fritted funnel rinsing with water until the eluent was colorless. The solid was further dried under high vacuum to afford a bright orange solid.

### Preparation of Intermediate 7

To a solution of compound 6 (74.9 mmol) in anhydrous EtOH (300 mL) and anhydrous DMF (75 mL) was slowly added sodium methoxide (30% w/w) (375 mmol) at room temperature under Argon atmosphere. After the addition, the solution was warmed to 60° C. for 2 hours. After cooling to ambient temperature, the solution was transferred to an Erlenmyer flask or tall beaker through dilution with water (700 mL) and then acidified with saturated citric acid. The resulting precipitate was collected on a sintered funnel rinsing with water. The crude product was purified by recrystallization in hot EtOH to afford a brown solid.

### Preparation of Compounds of Formula (II)

To a solution of intermediate 7 (1.00 mmol) in anhydrous pyridine (2.0 mL) was added an acid chloride of structure 8 (2.50 mmol) or the in situ formed mixed anhydride at room temperature. After stirring for 2-3 hours, the solution was diluted with 3M NaOH (6.0 mL) and stirred for another hour. The deep amber solution was transferred to an Erlenmeyer flask or beaker through dilution with water (100 mL) and then acidified with saturated citric acid. The resulting precipitate was collected on a sintered funnel rinsing with water. The crude product was further purified either by preparatory HPLC, or by recrystallization in hot ethanol or a mixture of hot ethanol and chloroform. Compounds B, C, D, K, L, U, AB. AC, AD, AE, AF, AG and AI were synthesized as described in Scheme 2.

### Compound D: (E)-N-[4-(6-Cyano-1-hydroxy-1H-benzoimidazol-2-yl)-phenyl]-3-(4-methylsulfanyl-phenyl)-acrylamide

¹H NMR (300 MHz, DMSO-d₆): δ 10.34 (s, 1H), 8.26 (d, J=8.4 Hz, 2H), 7.67 (d, J=8.7 Hz, 2H), 7.59-7.48 (m, 4H), 8.07 (s, 1H), 7.33-7.27 (m, 4H), 6.82 (d, J=15.6 Hz, 1H), 2.50 (s, 3H). MS (ESI, positive): calcd, 426.50; found, [M+1]⁺=427.10.

### Compound U: (E)-3-(4-Acetyl-phenyl)-N-[4-(1-hydroxy-6-methylsulfanyl-1H-benzoimidazol-2-yl)-phenyl]-acrylamide

¹H NMR (300 MHz, DMSO-d₆): δ 10.59 (s, 1H), 8.35 (d, J=8.7 Hz, 2H), 7.98 (d, J=8.1 Hz, 2H), 7.75-7.71 (m, 4H), 7.65 (d, J=15.6 Hz, 1H), 7.36 (d, J=8.4 Hz, 1H), 7.07-7.05 (m, 1H), 7.01 (s, 1H), 6.94 (dd, J=1.8, 8.4 Hz, 1H), 2.59 (s, 3H), 2.35 (s, 3H). MS (ESI, positive): calcd, 443.52; found, [M+1]⁺=444.20.

### Compound AB: (E)-N-[4-(6-Acetyl-1-hydroxy-1H-benzoimidazol-2-yl)-phenyl]-3-(4-fluoro-phenyl)-acrylamide

¹H NMR (300 MHz, DMSO-d₆): δ 10.53 (s, 1H), 8.30 (d, J=8.7 Hz, 2H), 8.13 (s, 1H), 7.93-7.87 (m, 3H), 7.74-7.71 (m, 3H), 7.64 (d, J=15.9 Hz, 1H), 7.30 (t, J=8.7 Hz, 2H), 7.82 (d, J=15.6 Hz, 1H), 2.67 (s, 3H). MS (ESI, positive): calcd, 415.42; found, [M+1]⁺=416.15.

### Compound AC: (E)-N-[4-(6-Fluoro-1-hydroxy-1H-benzoimidazol-2-yl)-phenyl]-3-(4-fluoro-phenyl)-acrylamide

¹H NMR (300 MHz, DMSO-d₆): δ 10.47 (s, 1H), 8.21 (d, J=8.4 Hz, 2H), 7.93 (d, J=8.4 Hz, 2H), 7.73-7.60 (m, 4H), 7.31 (qr, J=8.7 Hz, 3H), 7.08 (t, J=9.6 Hz, 1H), 6.81 (d, J=15.6 Hz, 1H). MS (ESI, positive): calcd, 391.38; found, [M+1]⁺=392.15.

### Compound AD: (E)-3-(4-Fluoro-phenyl)-N-[4-(1-hydroxy-6-methanesulfonyl-1H-benzoimidazol-2-yl)-phenyl]-acrylamide

¹H NMR (300 MHz, DMSO-d₆): δ 10.53 (s, 1H), 8.32 (d, J=8.4 Hz, 2H), 8.05 (s, 1H), 7.92 (d, J=8.4 Hz, 2H), 7.86 (d, J=8.7 Hz, 1H), 7.81-7.69 (m, 3H), 7.64 (d, J=15.6 Hz, 1H), 7.30 (t, J=8.4 Hz, 2H), 6.82 (d, J=15.6 Hz, 1H), 3.27 (s, 3H). MS (ESI, positive): calcd, 451.48; found, [M+1]⁺=452.15.

### Compound AE: (E)-3-(4-Fluoro-phenyl)-N-[4-(1-hydroxy-1H-benzoimidazol-2-yl)-phenyl]-acrylamide

¹H NMR (300 MHz, DMSO-d₆): δ 10.58 (s, 1H), 8.25 (d, J=8.4 Hz, 2H), 7.94 (d, J=8.4 Hz, 2H), 7.74-7.63 (m, 5H), 7.43-7.37 (m, 2H), 7.30 (t, J=8.7 Hz, 2H), 6.83 (d, J=15.6 Hz, 1H). MS (ESI, positive): calcd, 373.39; found, [M+1]⁺=374.15.

### Compound AF: (E)-3-(4-Fluoro-phenyl)-N-[4-(1-hydroxy-6-trifluoromethyl-1H-benzoimidazol-2-yl)-phenyl]-acrylamide

¹H NMR (300 MHz, DMSO-d₆): δ 10.54 (s, 1H), 8.30 (d, J=8.7 Hz, 2H), 7.92 (d, J=9.0 Hz, 2H), 7.87-7.83 (m, 2H), 7.71 (dd, J=5.4, 8.7 Hz, 2H), 7.69 (d, J=15.6 Hz, 1H), 7.56 (dd, J=1.5, 8.7 Hz, 1H), 7.29 (t, J=8.7 Hz, 2H), 6.82 (d, J=15.6 Hz, 1H). MS (ESI, positive): calcd, 441.38; found, [M+1]⁺=442.15.

### Compound AI: 2-{4-[(E)-3-(4-Fluoro-phenyl)-acryloylamino]-phenyl}-3-hydroxy-3H-benzoimidazole-5-carboxylic acid

¹H NMR (300 MHz, DMSO-d₆, a drop of 3M NaOH in D₂O): δ 8.44 (d, J=8.7 Hz, 2H), 7.97 (d, J=1.5 Hz, 1H), 7.57-7.46 (m, 5H), 7.19-7.12 (m, 4H), 6.74 (d, J=15.9 Hz, 1H), Protons for —COOH, —NOH, —NHCO are not seen in 3M NaOH in D₂O. MS (ESI, positive): calcd, 417.40; found, [M+1]⁺=418.10.

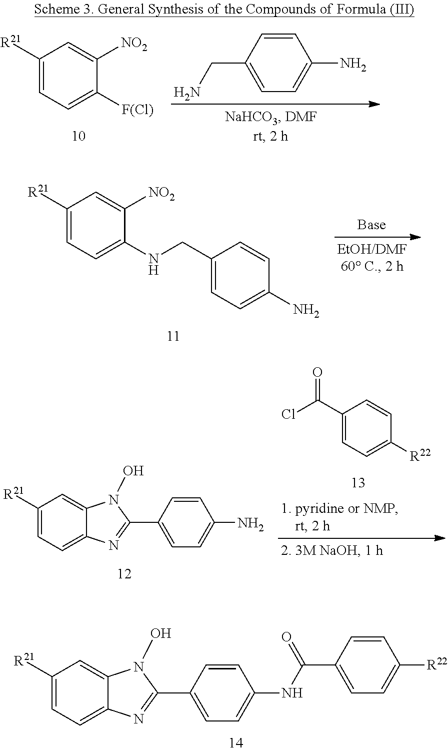

### Preparation of Intermediate 11

To a solution of 4-aminobenzyl amine (225 mmol) and powdered NaHCO₃ (1125 mmol) in anhydrous DMF (300 mL) at was added a substituted 2-nitrofluoro or 2-nitrochloro dibenzene (10) (150 mmol) dropwise at room temperature. After 2 hours, the solution was slowly diluted with water (1000 mL) to precipitate the product 11, which was collected on a fritted funnel rinsing with water until the eluent was colorless. The solid was further dried under high vacuum to afford a bright orange solid.

### Preparation of Intermediate 12

To a solution of compound II (74.9 mmol) in anhydrous EtOH (300 mL) and anhydrous DMF (75 mL) was slowly added sodium methoxide (30% w/w) (375 mmol) at room temperature under Argon atmosphere. After the addition, the solution was warmed to 60° C. for 2 hours. After cooling to ambient temperature, the solution was transferred to an Erlenmyer flask or tall beaker through dilution with water (700 mL) and then acidified with saturated citric acid. The resulting precipitate was collected on a sintered funnel rinsing with water. The crude product was purified by recrystallization in hot EtOH to afford a brown solid.

### Preparation of Compounds of Formula (III)

To a solution of intermediate 12 (1.00 mmol) in anhydrous pyridine (2.0 mL) was added an acid chloride of structure 13 (2.50 mmol) or the in situ formed mixed anhydride at room temperature. After stirring for 2-3 hours, the solution was diluted with 3M NaOH (6.0 mL) and stirred for another hour. The deep amber solution was transferred to an Erlenmeyer flask or beaker through dilution with water (100 mL) and then acidified with saturated citric acid. The resulting precipitate was collected on a sintered funnel rinsing with water. The crude product was further purified either by preparatory HPLC, or by recrystallization in hot ethanol or a mixture of hot ethanol and chloroform. Compounds E and F were synthesized as described in Scheme 3.

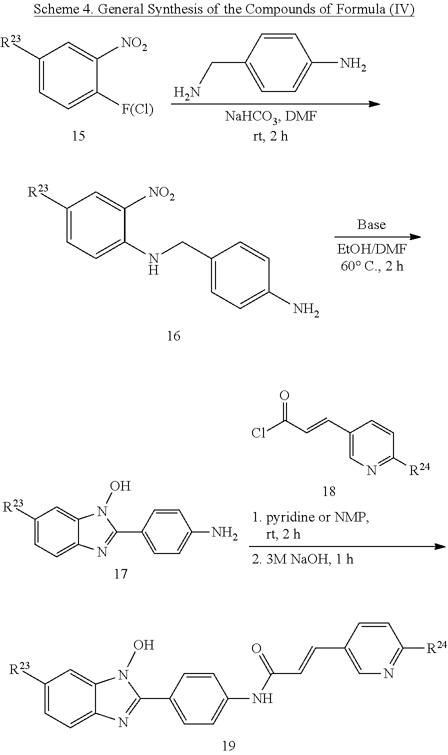

### Preparation of Intermediate 16

To a solution of 4-aminobenzyl amine (225 mmol) and powdered NaHCO₃ (1125 mmol) in anhydrous DMF (300 mL) at was added a substituted 2-nitrofluoro or 2-nitrochloro dibenzene (15) (150 mmol) dropwise at room temperature. After 2 hours, the solution was slowly diluted with water (1000 mL) to precipitate the product 16, which was collected on a fritted funnel rinsing with water until the eluent was colorless. The solid was further dried under high vacuum to afford a bright orange solid.

### Preparation of Intermediate 17

To a solution of compound 16 (74.9 mmol) in anhydrous EtOH (300 mL) and anhydrous DMF (75 mL) was slowly added sodium methoxide (30% w/w) (375 mmol) at room temperature under Argon atmosphere. After the addition, the solution was warmed to 60° C. for 2 hours. After cooling to ambient temperature, the solution was transferred to an Erlenmyer flask or tall beaker through dilution with water (700 mL) and then acidified with saturated citric acid. The resulting precipitate was collected on a sintered funnel rinsing with water. The crude product was purified by recrystallization in hot EtOH to afford a brown solid.

### Preparation of Compounds of Formula (IV)

To a solution of intermediate 17 (1.00 mmol) in anhydrous pyridine (2.0 mL) was added an acid chloride of structure 18 (2.50 mmol) or the in situ formed mixed anhydride at room temperature. After stirring for 2-3 hours, the solution was diluted with 3M NaOH (6.0 mL) and stirred for another hour. The deep amber solution was transferred to an Erlenmeyer flask or beaker through dilution with water (100 mL) and then acidified with saturated citric acid. The resulting precipitate was collected on a sintered funnel rinsing with water. The crude product was further purified either by preparatory HPLC, or by recrystallization in hot ethanol or a mixture of hot ethanol and chloroform. Compounds A, G, H and Z were synthesized as described in Scheme 4.

### Compound A: (E)-3-(6-Acetyl-pyridin-3-yl)-N-[4-(6-cyano-1-hydroxy-1H-benzoimidazol-2-yl)-phenyl]-acrylamide

¹H NMR (300 MHz, DMSO-d₆): δ 10.69 (s, 1H), 8.97 (d, J=1.2 Hz, 1H), 8.31 (d, J=8.7 Hz, 2H), 8.23 (dd, J=1.8, 8.1 Hz, 1H), 8.07 (s, 1H), 8.01 (d, J=8.1 Hz, 1H), 7.92 (d, J=8.7 Hz, 2H), 7.79 (d, J=8.7 Hz, 1H), 7.75 (d, J=15.9 Hz, 1H), 7.60 (dd, J=1.2, 8.4 Hz, 1H), 7.07 (d, J=15.9 Hz, 1H), 2.65 (s, 3H). MS (ESI, positive): calcd, 423.43; found, [M+1]⁺=424.20.

### Compound H: (E)-N-[4-(6-Cyano-1-hydroxy-1H-benzoimidazol-2-yl)-phenyl]-3-(6-methylsulfanyl-pyridin-3-yl)-acrylamide

¹H NMR (300 MHz, DMSO-d₆): δ 10.56 (s, 1H), 8.68 (s, 1H), 8.29 (d, J=7.8 Hz, 2H), 8.08 (s, 1H), 7.91 (d, J=7.8 Hz, 3H), 7.80 (d, J=8.1 Hz, 1H), 7.65-7.60 (m, 2H), 7.40 (d, J=8.1 Hz, 1H), 6.88 (d, J=15.6 Hz, 1H), 2.55 (s, 3H). MS (ESI, positive): calcd, 427.49; found, [M+1]⁺=428.15.

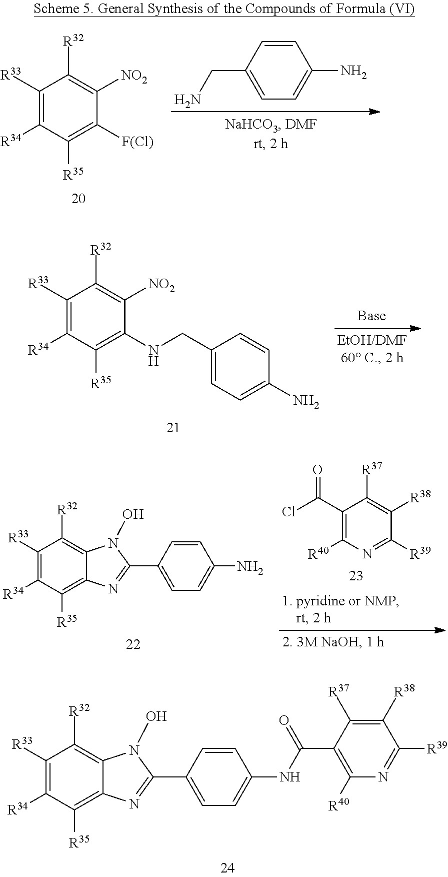

### Preparation of Intermediate 21

To a solution of 4-aminobenzyl amine (225 mmol) and powdered NaHCO₃ (1125 mmol) in anhydrous DMF (300 mL) at was added a substituted 2-nitrofluoro or 2-nitrochloro dibenzene (20) (150 mmol) dropwise at room temperature. After 2 hours, the solution was slowly diluted with water (1000 mL) to precipitate the product 21, which was collected on a fritted funnel rinsing with water until the eluent was colorless. The solid was further dried under high vacuum to afford a bright orange solid.

### Preparation of Intermediate 22

To a solution of compound 21 (74.9 mmol) in anhydrous EtOH (300 mL) and anhydrous DMF (75 mL) was slowly added sodium methoxide (30% w/w) (375 mmol) at room temperature under Argon atmosphere. After the addition, the solution was warmed to 60° C. for 2 hours. After cooling to ambient temperature, the solution was transferred to an Erlenmyer flask or tall beaker through dilution with water (700 mL) and then acidified with saturated citric acid. The resulting precipitate was collected on a sintered funnel rinsing with water. The crude product was purified by recrystallization in hot EtOH to afford a brown solid.

### Preparation of Compounds of Formula (VI)

To a solution of intermediate 22 (1.00 mmol) in anhydrous pyridine (2.0 mL) was added an acid chloride of structure 23 (2.50 mmol) or the in situ formed mixed anhydride at room temperature. After stirring for 2-3 hours, the solution was diluted with 3M NaOH (6.0 mL) and stirred for another hour. The deep amber solution was transferred to an Erlenmeyer flask or beaker through dilution with water (100 mL) and then acidified with saturated citric acid. The resulting precipitate was collected on a sintered funnel rinsing with water. The crude product was further purified either by preparatory HPLC, or by recrystallization in hot ethanol or a mixture of hot ethanol and chloroform. Compounds I, M, O, P, S, W, X and Y were synthesized as described in Scheme 5.

### Compound M: N-[4-(6-Cyano-1-hydroxy-1H-benzoimidazol-2-yl)-phenyl]-6-pyrazol-1-yl-nicotinamide

¹H NMR (300 MHz, DMSO-d₆): δ 10.63 (s, 1H), 9.04 (d, J=2.4 Hz, 1H), 8.91 (d, J=8.1 Hz, 2H), 8.72 (d, J=2.4 Hz, 1H), 8.53 (dd, J=2.1, 8.4 Hz, 1H), 8.05 (d, J=8.7 Hz, 1H), 7.91 (s, 1H), 7.85 (d, J=8.7 Hz, 2H), 7.72 (s, 1H), 7.44 (d, J=8.4 Hz, 1H), 7.18 (dd, J=1.5, 8.4 Hz, 1H), 6.64 (t, J=2.4 Hz, 1H). MS (ESI, positive): calcd, 421.42; found, [M+1]⁺=422.20.

### Compound P: N-[4-(6-Cyano-1-hydroxy-1H-benzoimidazol-2-yl)-phenyl]-6-morpholin-4-yl-nicotinamide

¹H NMR (300 MHz, DMSO-d₆): δ 10.25 (s, 1H), 8.79 (d, J=2.4 Hz, 1H), 8.75 (br d, J=7.2 Hz, 2H), 8.15 (dd, J=2.4, 9.0 Hz, 1H), 7.82 (d, J=9.0 Hz, 2H), 7.63 (br s, 1H), 7.44 (d, J=8.4 Hz, 1H), 7.19 (dd, J=1.8, 8.4 Hz, 1H), 6.90 (d, J=9.3 Hz, 1H), 3.71-3.67 (m, 4H), 3.62-3.57 (m, 4H). MS (ESI, positive): calcd, 440.46; found, [M+1]⁺=441.25.

### Compound W: N-[4-(1-Hydroxy-6-nitro-1H-benzoimidazol-2-yl)-phenyl]-2-methyl-6-trifluoromethyl-nicotinamide

¹H NMR (300 MHz, DMSO-d₆): δ 10.94 (s, 1H), 8.37 (dd, J=3.3, 5.7 Hz, 3H), 8.23 (d, J=7.8 Hz, 1H), 8.13 (dd, J=2.1, 9.0 Hz, 1H), 7.96 (d, J=8.7 Hz, 2H), 7.91 (d, J=7.8 Hz, 1H), 7.84 (d, J=9.0 Hz, 1H), 2.66 (s, 3H). MS (ESI, positive): calcd, 457.37; found, [M+1]⁺=458.20.

### Compound X: N-[4-(1-Hydroxy-6-nitro-1H-benzoimidazol-2-yl)-phenyl]-6-(2,2,2-trifluoro-ethoxy)-nicotinamide

¹H NMR (300 MHz, DMSO-d₆): δ 10.64 (s, 1H), 8.83 (d, J=2.4 Hz, 1H), 8.37-8.34 (m, 4H), 8.13 (dd, J=2.4, 9.0 Hz, 1H), 8.01 (d, J=9.0 Hz, 2H), 7.82 (d, J=9.0 Hz, 1H), 7.16 (d, J=8.7 Hz, 1H), 5.11 (qr, J=9.0 Hz, 2H). MS (ESI, positive): calcd, 473.37; found, [M+1]⁺=474.20.

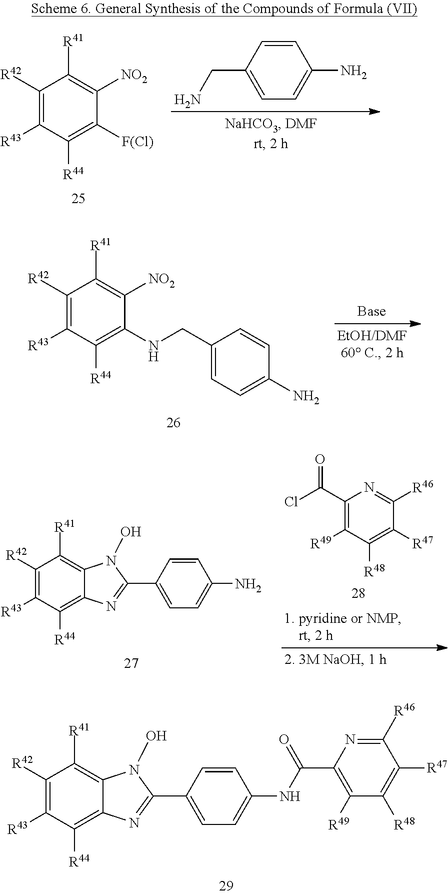

### Preparation of Intermediate 26

To a solution of 4-aminobenzyl amine (225 mmol) and powdered NaHCO₃ (1125 mmol) in anhydrous DMF (300 mL) at was added a substituted 2-nitrofluoro or 2-nitrochloro dibenzene (25) (150 mmol) dropwise at room temperature. After 2 hours, the solution was slowly diluted with water (1000 mL) to precipitate the product 26, which was collected on a fritted funnel rinsing with water until the eluent was colorless. The solid was further dried under high vacuum to afford a bright orange solid.

### Preparation of Intermediate 27

To a solution of compound 26 (74.9 mmol) in anhydrous EtOH (300 mL) and anhydrous DMF (75 mL) was slowly added sodium methoxide (30% w/w) (375 mmol) at room temperature under Argon atmosphere. After the addition, the solution was warmed to 60° C. for 2 hours. After cooling to ambient temperature, the solution was transferred to an Erlenmyer flask or tall beaker through dilution with water (700 mL) and then acidified with saturated citric acid. The resulting precipitate was collected on a sintered funnel rinsing with water. The crude product was purified by recrystallization in hot EtOH to afford a brown solid.

### Preparation of Compounds of Formula (VII)

To a solution of intermediate 27 (1.00 mmol) in anhydrous pyridine (2.0 mL) was added an acid chloride of structure 28 (2.50 mmol) or the in situ formed mixed anhydride at room temperature. After stirring for 2-3 hours, the solution was diluted with 3M NaOH (6.0 mL) and stirred for another hour. The deep amber solution was transferred to an Erlenmeyer flask or beaker through dilution with water (100 mL) and then acidified with saturated citric acid. The resulting precipitate was collected on a sintered funnel rinsing with water. The crude product was further purified either by preparatory HPLC, or by recrystallization in hot ethanol or a mixture of hot ethanol and chloroform. Compound N was synthesized as described in Scheme 6.

### Compound N: 6-Morpholin-4-yl-pyridine-2-carboxylic acid [4-(1-hydroxy-6-nitro-1H-benzoimidazol-2-yl)-phenyl]-amide

¹H NMR (300 MHz, DMSO-d₆): δ 10.24 (s, 1H), 8.93 (d, J=8.7 Hz, 2H), 8.24 (d, J=2.4 Hz, 1H), 7.91 (d, J=8.7 Hz, 2H), 7.86-7.76 (m, 2H), 7.46 (d, J=7.2 Hz, 1H), 7.41 (d, J=9.0 Hz, 1H), 7.11 (d, J=8.7 Hz, 1H), 3.76 (t, J=4.2 Hz, 4H), 3.62 (t, J=3.9 Hz, 4H). MS (ESI, positive): calcd, 460.45; found, [M+1]⁺=461.20.

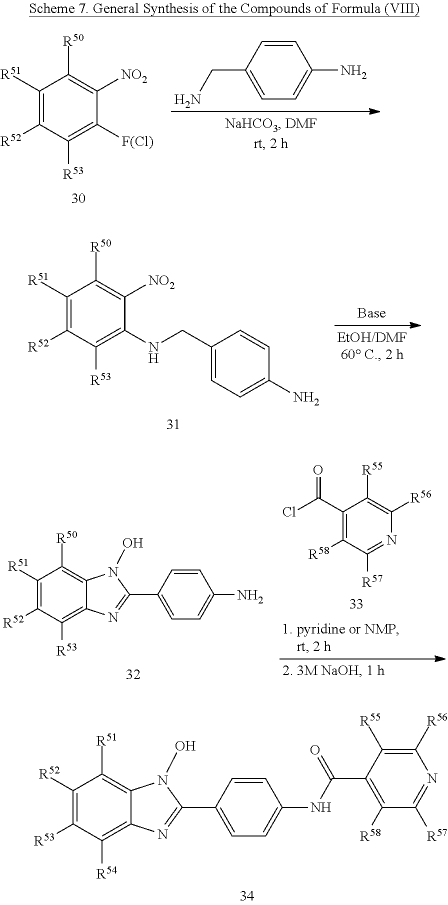

### Preparation of Intermediate 31

To a solution of 4-aminobenzyl amine (225 mmol) and powdered NaHCO₃ (1125 mmol) in anhydrous DMF (300 mL) at was added a substituted 2-nitrofluoro or 2-nitrochloro dibenzene (30) (150 mmol) dropwise at room temperature. After 2 hours, the solution was slowly diluted with water (1000 mL) to precipitate the product 31, which was collected on a fritted funnel rinsing with water until the eluent was colorless. The solid was further dried under high vacuum to afford a bright orange solid.

### Preparation of Intermediate 32

To a solution of compound 31 (74.9 mmol) in anhydrous EtOH (300 mL) and anhydrous DMF (75 mL) was slowly added sodium methoxide (30% w/w) (375 mmol) at room temperature under Argon atmosphere. After the addition, the solution was warmed to 60° C. for 2 hours. After cooling to ambient temperature, the solution was transferred to an Erlenmyer flask or tall beaker through dilution with water (700 mL) and then acidified with saturated citric acid. The resulting precipitate was collected on a sintered funnel rinsing with water. The crude product was purified by recrystallization in hot EtOH to afford a brown solid.

### Preparation of Compounds of Formula (VIII)

To a solution of intermediate 32 (1.00 mmol) in anhydrous pyridine (2.0 mL) was added an acid chloride of structure 33 (2.50 mmol) or the in situ formed mixed anhydride at room temperature. After stirring for 2-3 hours, the solution was diluted with 3M NaOH (6.0 mL) and stirred for another hour. The deep amber solution was transferred to an Erlenmeyer flask or beaker through dilution with water (100 mL) and then acidified with saturated citric acid. The resulting precipitate was collected on a sintered funnel rinsing with water. The crude product was further purified either by preparatory HPLC, or by recrystallization in hot ethanol or a mixture of hot ethanol and chloroform. Compound T was synthesized as described in Scheme 7.

### Example 2

### Development of Luminescence Assays

A quantitative chemiluminescence-based assay was used to measure the DNA binding activity of various MarA (AraC) family members. With this technique, biotinylated double-stranded DNA molecules (2 nM) were incubated with a MarA (AraC) protein (20 nM) fused to 6-histidine (6-His) residues in a streptavidin coated 96-well microtiter (white) plate (Pierce Biotechnology, Rockford, Ill.). Unbound DNA and protein was removed by washing and a primary monoclonal anti-6His antibody was subsequently added. A second washing was performed and a secondary HRP-conjugated antibody was then added to the mixture. Excess antibody was removed by a third wash step and a chemiluminescence substrate (Cell Signaling Technology, Beverly, Mass.) was added to the plate. Luminescence was read immediately using a Victor V plate reader (PerkinElmer Life Sciences, Wellesley, Mass.). Compounds that inhibited the binding of the protein to the DNA resulted in a loss of protein from the plate at the first wash step and were identified by a reduced luminescence signal. The concentration of compound necessary to reduce signal by 50% (IC₅₀) was calculated using serial dilutions of the inhibitory compounds. Also, single transcription factor modulators that affect different transcription factors were identified.

### Example 3

### In Vivo Activity of Select Transcription Factor Modulating Compounds in an Ascending Pyelonephritis Model of Infection

Using an animal model of ascending pyelonephritis caused by E. coli, transcription factor modulating compounds are judged for the ability to affect kidney infection. Previous studies using this urinary tract infection model have shown that E. coli mutants with a soxS gene deletion colonize the mouse kidney in numbers approximately 1-log fewer than the wild type strain. Groups of female CD1 mice (n=6) are diuresed and infected with E. coli UPEC strain C189 via intravesicular inoculation. Subsequently, mice are dosed with a transcription factor modulator (25 or 50 μg/ml), a control compound, e.g., SXT, or vehicle alone (0 mg/kg), via an oral route of administration at the time of infection and once a day for 4 days thereafter, to maintain a constant level of drug in the mice. After a 5-day period of infection and prior to sacrifice via CO₂/O₂ asphyxiation, a urine sample is taken by gentle compression of the abdomen. Following asphyxiation, the bladder and kidneys are removed aseptically. Urine volumes and individual organ weights are recorded, the organs are suspended in sterile PBS containing 0.025% Triton X-100, and then homogenized. Serial 10-fold dilutions of the urine samples and homogenates are plated onto McConkey agar plates to determine CFU/ml (CFU=colony forming units) of urine or CFU/gram of organ. Efficacy in these experiments is defined as a ≧2-log decrease in CFU/g organ.

### Example 4

### In Vitro Activity of Select Transcription Factor Modulating Compounds Against LcrF (VirF) from Y. pseudotuberculosis

The Y. pseudotuberculosis protein LcrF (also called VirF in Y. enterocolitica) regulates expression of a major virulence determinant, the type III secretion system (TTSS). The TTSS delivers toxins directly into host cells, and mutants that do not express the TTSS show dramatic attenuation of virulence in whole cell and animal models of infection. In order to determine the inhibition of LcrF-DNA binding by the transcription factor modulating compounds of the invention, the MarA (AraC) family member LcrF (VirF) was cloned, expressed and purified from Y. pseudotuberculosis. The purified protein was used in a cell-free system to monitor DNA-protein interactions in vitro, methods as in Example 2. The IC₅₀'s for inhibition of LcrF(VirF)-DNA binding by the compounds of the invention are summarized in Table 3 below. Compounds with excellent inhibition (less than 10 μM) are indicated with “***,” very good inhibition (greater than 10.0 and less than 25.0 μM) with “**,” good inhibition (greater than 25.0 μM and less than 50.0 μM) with “*” and weak to no inhibition (greater than 50 μM) with “--.”

TABLE 3

| Compound | IC₅₀(μM) | Compound | IC₅₀(μM) |
|---|---|---|---|
| A | * | S | — |
| B | — | T | — |
| C | ** | U | — |
| D | * | V | *** |
| E | — | W | — |
| F | — | X | * |
| G | ** | Y | ** |
| H | * | Z | — |
| I | * | AA | *** |
| J | *** | AB | * |
| K | — | AC | — |
| L | — | AD | — |
| M | — | AE | — |
| N | — | AF | — |
| O | — | AG | — |
| P | — | AH | ** |
| Q | * | AI | * |
| R | * | | |

### Example 5

### Inhibition of Y. pseudotuberculosis Cytotoxic Activity by Select Transcription Factor Modulating Compounds in a Whole Cell Assay

In order to demonstrate that the transcription factor modulating compounds of the invention inhibit LcrF(VirF)-dependent cytotoxicity of Y. pseudotuberculosis, select compounds were screened in a whole cell system, which are models of infection in which virulence is measured by bacterial cytotoxicity towards the host cell. In this assay, type III secretion, the process whereby cytotoxic proteins (Yops) are secreted from a bacterium into a host cell, in pathogenic Yersinia spp. is regulated by LcrF. Wild type Y. pseudotuberculosis are toxic toward J774 tissue culture cells whereas bacteria bearing a mutation in either yopJ (a Yop that inhibits eukaryotic signaling pathways) or lcrF are not. The cytotoxicity of wild type Y. pseudotuberculosis was exploited in order to screen compounds for their ability to penetrate the intact bacterial cell and prevent type III secretion by binding to an inactivating LcrF function.

The CytoTox 96® assay kit from Promega was used for this assay. Briefly, J774 macrophages were plated out at 2×10⁴ cells per well in 96-well plates on the day prior to infection. Yersinia pseudotuberculosis were grown overnight at 26° C. in 2×YT media and then diluted 1:25 or 1:40 the following morning into 2×YT supplemented with 20 mM MgCl₂ and 20 mM sodium oxalate. The cultures were grown for a further 90 min at 26° C. and then shifted to 37° C. for 90 minutes. The temperature shift and the sodium oxalate, which chelates calcium, lead to induction of LcrF expression. Later experiments also included the YPIIIpIB1ΔJ (YopJ mutant) and YPIIIpIB1ΔLcrF (LcrF mutant). YPIIIpIB1ΔJ is a YopJ deletion mutant and any cytotoxicity that is unrelated to YopJ (i.e. lps-mediated) will be seen with this strain. The OD600 was measured and the culture adjusted to an OD600 of 1.0. This should correspond to approximately 1.25×10⁹ cells/mL. Dilutions were prepared in DMEM (the J774 culture media) at different multiplicity of infections (MOIs), assuming J774 cell density of 2×10⁴. Yersinia pseudotuberculosis were added in 10 μl aliquots and cells were incubated at 37° C. either in a chamber with a CO₂ generating system, or later, in a tissue culture incubator with 5% CO₂ for 2 hours. Gentamicin was then added to a final concentration of 50 μg/ml and the incubations were continued either for a further 2-3 h or overnight. Controls were included for media alone, target cell spontaneous lysis, target cell maximum lysis and effector cell spontaneous lysis. For maximum lysis, triton X-100 was added to a final concentration of 0.8% 45 minutes prior to termination of the experiment. Supernatants containing released LDH were harvested following centrifugation at 1,000 rpm for 5 minutes. Supernatants were either frozen overnight or assayed immediately. 50 μl of supernatant was mixed with 50 μl fresh assay buffer and incubated in the dark for 30 minutes 50 μl of stop solution was added to each well and the plates were read at 490 nm. In Table 4 below, compounds that reduced Y. pseudotubercolosis cytotoxicity to 99-75% of untreated, wild type levels at 50 μg/mL are indicated with “**.” Compounds that reduced Y. pseudotubercolosis cytotoxicity to below ≧75% of untreated, wild type levels at 50 μg/mL are indicated with “*.” The percent cytotoxicity was measured relative to vehicle treated cells infected with wild type Y. pseudotuberculosis. Incubation with wild type Y. pseudotuberculosis yields ≧75% toxicity. This data illustrate that the compounds of the invention reduces the cytotoxicity of Y. pseudotuberculosis against the host cell. The reduced cytotoxicity correlates with reduced virulence.

TABLE 4

| Compound | % Cytotoxicity | Compound | % Cytotoxicity |
|---|---|---|---|
| A | * | S | ** |
| B | ** | T | * |
| C | ** | U | ** |
| D | ** | V | * |
| E | ** | W | ** |
| F | ** | X | ** |
| G | * | Y | * |
| H | * | Z | ** |
| I | * | AA | * |
| J | * | AB | ** |
| K | ** | AC | ** |
| L | ** | AD | ** |
| M | * | AE | ** |
| N | ** | AF | ** |
| O | ** | AG | ** |
| P | ** | AH | ** |
| Q | ** | AI | * |
| R | ** | | |

### Example 6

### Efficacy of Select Transcription Factor Modulating Compounds in a Y. pseudotuberculosis Pneumonia Model

The transcription factor modulating compounds of the invention that reduce Y. pseudotuberculosis cytotoxicity are then tested in lethal and sublethal murine Y. pseudotuberculosis murine models. Groups of 4 CD1 mice (7-8 week old males) are dosed subcutaneously with either vehicle or compound (25 mg/kg) 1 day prior to infection, at the time of infection, at 8 hours and then daily for 8 days following intranasal infection with approximately 120 CFU of wild type (WT, IP2666pIB1) or ΔLcRF (JMB155) Y. pseudotuberculosis. The percent loss of starting weight and percent survival of infected mice following treatment with a transcription factor modulating compound is monitored.

Another assay is performed in which groups of CD-1 mice are treated with a single subcutaneous dose of vehicle or LcrF inhibitor (25 mg/kg) one day prior to infection, at the time of infection, at 8 hours post infection, then once daily for a further 2 days. Mice are infected intranasally with 728 CFU of wild type (IP2666pIB1) or 752 CFU ΔLcrF (JMB155) Y. pseudotuberculosis. The mice are sacrificed 3 days post infection and serial dilutions of lung tissue homogenates are plated. This assay illustrates that treatment with select transcription factor modulating compounds can reduced bacterial burden in the lung and decreased mortality in these mouse models of pneumonia.

### Example 7

### In Vitro Activity of Select Transcription Factor Modulating Compounds Against ExsA from Pseudomonas aeruginosa

ExsA regulates the expression of a major virulence determinant, the type III secretion system (TTSS). It has been shown that mutants that lack the exsA gene do not express the TTSS and these mutants show dramatically reduced virulence in whole cell assays and animal models of P. aeruginosa infection. The vast majority of clinical P. aeruginosa strains have the TTSS and expression of the TTSS is correlated with increased severity of disease in clinical pneumonia cases, including ventilator associated pneumonia. Several transcription factor modulating compounds with high activity against LcrF also showed good inhibition of ExsA-DNA binding in vitro. The MarA (AraC) family member ExsA was cloned, expressed and purified from P. aeruginosa. The purified protein was used in a cell-free system to monitor DNA-protein interactions in vitro, methods as in Example 2. The IC₅₀'s for inhibition of ExsA-DNA binding by the compounds of the invention are summarized in Table 5 below. Compounds with excellent inhibition (less than 10 μM) are indicated with “***,” very good inhibition (greater than 10.0 and less than 25.0 μM) with “**,” good inhibition (greater than 25.0 μM and less than 50 μM) with “*” and weak or no inhibition (greater than 50 μM) with

TABLE 5

| Compound | IC₅₀(μM) | Compound | IC₅₀(μM) |
|---|---|---|---|
| A | — | O | — |
| B | ** | P | *** |
| C | ** | Q | * |
| D | *** | R | * |
| E | — | S | * |
| F | ** | T | — |
| G | *** | U | ** |
| H | *** | V | *** |
| I | * | W | — |
| J | *** | X | * |
| K | — | Y | ** |
| L | ** | Z | — |
| N | ** | | |

### Example 8

### Inhibition of P. aeruginosa Cytotoxicity by Select Transcription Factor Modulating Compounds in a Whole Cell Assay

Transcription factor modulating compounds that exhibited measurable inhibition of ExsA-DNA binding, as described in Example 7, were screened for inhibition of ExsA-dependent P. aeruginosa cytotoxicity to macrophages in a whole cell system, which is a model of infection in which virulence is measured by bacterial cytotoxicity towards the host cell. In pathogenic P. aeruginosa, type III secretion is regulated by ExsA. Type III secretion is the process in which cytotoxic proteins (ExoU, ExoT, etc.) are secreted from a bacterium into a host cell. Wild type P. aeruginosa are toxic toward J774 tissue culture cells whereas bacteria bearing a mutation in exsA are not. In this example, the cytotoxicity of wild type P. aeruginosa was exploited to screen compounds for their ability to penetrate the intact bacterial cell and prevent type III secretion by binding to an inactivating ExsA function.

The CytoTox 96® assay kit from Promega was used for this assay. Briefly, J774 macrophage-like cells were plated out at 5×10⁴ cells per well in 96-well plates on the day prior to infection. P. aeruginosa were grown overnight at 37° C. in Luria Broth and then diluted 1:25 in MinS, a minimal salt media containing the calcium chelator trisodium nitriloacetate. Experiments also included the WT ExsA mutants, in which the entire exsA coding sequence has been deleted. Mar inhibitors were added to the MinS cultures at a concentration of 50 μg/mL and the cultures were grown for a further 3 hours at 37° C. The shift to a calcium free media leads to induction of ExsA expression. Cultures were grown to an OD600 of 1.0, approximately 1×10⁹ cells/mL. Dilutions were prepared in DMEM (the J774 culture media) at different multiplicity of infections (MOIs), assuming J774 cell density of 5×10⁴. Media in the J774 cell wells was replaced with DMEM containing 50 μg/mL of Mar inhibitors. P. aeruginosa were added to J774 cells in 10 μl aliquots, plates were centrifuged at 1,000 rpm for 5 minutes to synchronize infection and then incubated in a tissue culture incubator with 5% CO₂ for 2 h. Controls were included for media alone, target cell spontaneous lysis, target cell maximum lysis, and Mar inhibitors with J774 cells alone. For target cell maximum lysis, 10 μl of the CytoTox 96® assay kit lysis solution was added to untreated J774 cells 30 minutes prior to termination of the experiment. Supernatants containing released LDH were harvested following centrifugation at 1,000 rpm for 5 minutes. Supernatants were stored frozen overnight or assayed immediately. 50 μl of supernatant was mixed with 50 μl fresh LDH substrate solution and incubated in the dark for 30 minutes. 50 μl of stop solution was added to each well and the plates were read at 490 nm. In Table 6 below, compounds that reduced P. aeruginosa cytotoxicity to 99-75% of untreated, wild type levels at 50 mg/mL are indicated with “*.” Compounds that reduced P. aeruginosa cytotoxicity below 75% of untreated, wild type levels at 50 mg/mL are indicated with “**.” The percent cytotoxicity was relative to vehicle treated cells infected with wild type P. aeruginosa. Incubation with wild type P. aeruginosa yielded ≧75% toxicity. In addition, an exsA null mutant was completely non-cytotoxic. This data illustrate that the compounds of the invention reduces the cytotoxicity of P. aeruginosa against the host cell. The reduced cytotoxicity correlates with reduced virulence.

TABLE 6

| Compound | % Cytotoxicity | Compound | % Cytotoxicity |
|---|---|---|---|
| A | ** | S | * |
| B | * | T | * |
| C | * | U | * |
| D | * | V | ** |
| E | * | W | * |
| F | * | X | * |
| G | ** | Y | * |
| H | ** | Z | * |
| I | * | AA | ** |
| J | ** | AB | * |
| K | * | AC | * |
| L | * | AD | * |
| M | * | AE | * |
| N | * | AF | * |
| O | * | AG | * |
| P | * | AH | * |
| Q | * | AI | * |
| R | * | | |

### Example 9

### Efficacy of Select Transcription Factor Modulating Compounds in a Lethal P. aeruginosa Pneumonia Model

Transcription factor modulating compounds that substantially inhibited P. aeruginosa cytotoxicity are tested in a lethal model of murine acute pneumonia. In this model, infection with ˜1×10⁶ CFU of wild type bacteria causes >90% mortality within 48-72 hours, whereas mice infected with the same number of an exsA null mutant bacteria survive indefinitely. The efficacy of select transcription factor modulating compounds are tested in vivo for their efficacy against P. aeruginosa PA103 in a mouse model of pneumonia (10⁶ organisms inoculated intranasally). The compounds are administered IP at 25 mg/kg at −18, −1, 2, 5, 20, 26 and 44 hours post-infection and mortality was assessed at various times post infection. This assay measures percent survival rate of treated mice over a period of time post infection as compared to the untreated group.

### Example 10

### In Vitro Activity of Select Transcription Factor Modulating Compounds Against SoxS from E. coli

The E. coli protein SoxS regulates genes involved in bacterial resistance to oxidative stress and antibiotics. SoxS is required for full E. coli virulence in a murine ascending pyelonephritis model. In order to determine the inhibition of SoxS-DNA binding by the transcription factor modulating compounds of the invention, the MarA (AraC) family member SoxS was cloned, expressed and purified from E. coli. The purified protein was used in a cell-free system to monitor DNA-protein interactions in vitro, methods as in Example 2. The IC₅₀'s for inhibition of SoxS-DNA binding by the compounds of the invention are summarized in Table 7 below. Compounds with excellent inhibition (less than 10 μM) are indicated with “***,” very good inhibition (greater than 10.0 and less than 25.0 μM) with “**,” good inhibition (greater than 25.0 μM and less than 50 μM) with “*” and weak or no inhibition (greater than 50 μM) with “--.”

TABLE 7

| Compound | IC50(μM) |
|---|---|
| C | * |
| D | ** |
| G | — |
| H | — |
| J | *** |
| M | — |
| P | — |

### Example 11

### E. coli Biofilm Assay

The biofilm assay screens test compounds for their ability to inhibit bacteria from forming a biofilm.

### Materials:

The M9 media (“M9”) contains M9, casamino acids, and glucose. The test compound is dissolved in 10 mg/mL DMSO stock solution.

### Method:

### Preparation of Inoculum

Inoculum is started the day of the experiment by adding a colony or glycerol stock stab to 2 mL M9. The tube is placed in the 37° C. shaker incubator for approximately 4-6 hours. This inoculum is referred to as the “Starter inoculum.” The inoculum is then removed from the shaker incubator and diluted to 1×10⁶ cells/mL in M9.

### Preparation of Controls

Typically, there are eight of each control, including a positive and negative control. For both the positive and negative controls, 2.5 μL of DMSO is added to 200 μL of M9. 25 μL of the diluted DMSO is added to 50 μL of M9 in the assay plate.

### Preparation of Test Compounds

The test compounds are screened at 20 μg/mL. 2.5 μL of the test compound are taken from a plate containing 10 mg/mL stock and added to 200 μL of M9 and mixed. 25 μL of the diluted test compound is added to 50 μL of M9 in the assay plate. The resulting concentration of the test compound is 40 μg/mL

### Preparation of Plate

75 μL of the inoculum at 1×10⁶ cells/mL is added to each well containing compound and the positive controls. 75 μM9 is added to the negative controls. The final concentration of the test compound is 20 μg/mL and the final concentration of the inoculum is 2×10⁵ cells/mL. The plates are then placed in a microplate reader (Wallac Victor²V) and read OD₅₃₅ (“Initial growth reading”). The plates are then placed in an incubator overnight at 35° C. In the morning, the plates are read in a microplate reader at OD₅₃₅ (“Final growth reading.”)

### Addition of Crystal Violet

The inoculum is then removed from the wells and the plates are washed several times with tap water. 150 μL of Crystal Violet (0.02% Crystal Violet dissolved in water) is then added to each well.

### Addition of Ethanol

The crystal violet is then removed and the plates are washed several times with tap water. 150 μL of ethanol is then added to each well, after mixing. The plates are then placed in a microplate reader and read the OD₅₃₅. This is referred to as the “Crystal Violet” reading.

### Data Analysis

To determine whether a test compound inhibits growth, the initial growth reading is subtracted from the final growth reading (“Subtracted Growth”). The same is done for the positive controls and averaged. The % inhibition of growth is determined by the following formula:

100−(100*Subtracted growth of sample/Average growth of Pos Controls)

To determine whether a test compound inhibits biofilm formation, the percent inhibition of biofilm formation is determined using the following formula:

100−(100*Crystal Violet read of sample/Average crystal violet read of Pos Controls)

### Example 12

### LANCE Screening Assay for Select Transcription Factor Modulating Compound Inhibitors of SoxS, ExsA, VirF and SlyA DNA-binding

This example describes a method for the identification of test compounds that inhibit the interactions of purified transcription factor such as SoxS, ExsA and/or VirF with a target DNA sequence in an in vitro system.

### Materials

The 6His-tagged SoxS, ExsA and VirF are purified according to respective protocol. The N-term-biotinylated double-stranded DNA has a sequence of CCG ATT TAG CAA AAC GTG GCA TCG GTC (SEQ ID NO. 1). The antibody used is the LANCE Eu-labeled anti-6×His Antibody (Eu-αHis) (Perkin Elmer cat #AD0110) which has at least 6 Europium molecules per antibody. Streptavidin conjugated to SureLight™-Allophycocyanin (SA-APC) is obtained from Perkin Elmer (cat #CR130-100). The assay buffer contains 20 mM Hepes pH 7.6, 1 mM EDTA, 10 mM (NH₄)₂SO₄, and 30 mM KCl, and 0.2% Tween-20.

### Method

The plates or vials of the compounds to be tested are thawed. These stocks are at a concentration of 10 mg/ml in DMSO. The solutions are allowed to thaw completely, and the plates are briefly shaken on the Titermix to redissolve any precipitated compound. Thawed aliquots of SoxS, ExsA and VirF protein from the stock stored at −80° C. and 1M stock of dithiothreitol stored at −20° C. are then placed on ice.

Dilutions at 1:100 of the compounds are made into a fresh 96-well polystyrene plate. The dilutions are prepared with 100% DMSO to give a final concentration of 100 μg/ml solutions. The dilutions are vortexed on a Titermix.

Fresh DTT is added to 25-50 mL of assay buffer to produce a 1 mM final concentration. Next, 90 μl of assay buffer is added to each of the 10 μl protein aliquots, and the solution is mixed by pipetting. These proteins are diluted to give the required amount of each of the diluted proteins, resulting in 20 μl of diluted protein per well. In preparing the solutions, 20% excess is made to allow enough for control wells. Typically, depending on the protein preps and the initial binding curves that are performed, 1000-2000 fmoles of each protein is required per well. The diluted protein solutions are the placed on ice.

Three tests plates per plate of compound (for SoxS, ExsA and VirF) are prepared. Using a multichannel pipet, 5 μl of the compound is added to each well. 5 μl of DMSO is added to the blank and control wells, and 5 μl of the control inhibitor is added to the respective wells.

Using the multichannel pipet, 20 μl of protein is added to all wells except those designated “blank”. To these blank wells, 20 μl of assay buffer is added. The plates are covered with a plate sealer and incubated at room temperature, shaking on the Titermix, for 30 minutes.

Next, the DNA solution is prepared, with enough for at least 20% more wells than were tested. 15 μl (0.4 fmoles) is added per well. Then the DNA is diluted in assay buffer, and vortexed briefly to mix. The plate sealer is removed, and 15 μl of DNA solution is added to all of the wells. The plates are then resealed, and returned to the Titermix for a further 30 minutes.

After 25 minutes, the antibody solution is prepared. 0.4 fmoles of SA-APC and 0.125 fmoles of Eu-αHis are added per well in a total volume of 10 μl. Amounts are prepared sufficient for at least 20% excess. The plate sealer is the removed and 10 μl of antibody solution is added to every well. The plates are subsequently resealed, placed on the Titermix, and covered with aluminum foil. The plates are mixed for 1 hour. The plates are then read on the Wallac Victor V, using the LANCE 615/665 protocol.

### Data Processing

For each plate, the mean control (i.e., signal from protein and DNA without inhibition), mean blank (background signal without protein) and mean inhibitor (P001407) LANCE₆₆₅ counts are determined. The percentage inhibition by each molecule (each test well) is then determined according to the following equation:

% Inhibition=100−(((test−mean blank)/(mean control−mean blank)*100)

Compounds that gave 40% or greater inhibition are identified as hits and screened again for IC₅₀.

### IC₅₀ Screening

The protocol used is identical to that outlined above, except that only 10 compounds are assayed per plate. The testing concentrations start at 10 μg/ml and are diluted two-fold from 10 to 0.078 μg/ml.

### IC₅₀ Data Processing

Percent inhibition is calculated as shown above. Percent inhibition is then plotted vs. log (conc. inhibitor) using Graphpad Prism software.

### Example 13

### Assay to Detect Direct DNA Binding by Test Compounds

To assess non-specific DNA binding by test compounds, the effects of transcription factor modulating compounds on the migration of a supercoiled plasmid DNA during agarose gel electrophoresis are tested. Compounds are diluted in DMSO with 0.4% ethanolamine to a concentration of 1 mg/mL. In a clear 96 well plate, 2 μL of 1 mg/mL compound solution is added to 18 μL assay buffer (20 mM Hepes, pH 7.6, 10 mM (NH₄)₂SO₄, 30 mM KCl, 1 mM EDTA, 0.2% Tween-20) containing 100 ng of pET-15b supercoiled plasmid DNA. Final compound test concentration is therefore 100 μg/mL. The plate is incubated at room temperature for 30 minutes. The plate is photographed under white light and then under UV light to assess fluorescence, a property of DNA intercalators. Ten microliters of compound and DNA solutions are loaded onto a 0.8% agarose gel and electrophoresed. The gel is then stained with ethidium bromide and photographed. Control tests with drug vehicle (DMSO with 0.4% ethanolamine) does not cause fluorescence under UV light or effect DNA migration through the gel. In contrast, tests with 100 μg/mL of positive control Hoechst 33342 dye, a known DNA intercalator, results in bright fluorescence under UV light and a faint smear on the gel rather than the discrete bands seen when unperturbed DNA is electrophoresed.

### Example 14

### In Vitro Activity of Select Transcription Factor Modulating Compounds Against SlyA from S. typhimurium enterica serovar Typhimurium

Test compounds are further screened for specificity toward MarA (AraC) family proteins by testing for inhibition of SlyA-DNA binding in vitro. SlyA is a member of a different DNA-binding protein superfamily (the MarR family) and is not related to members of the MarA protein family. Compounds with specific inhibition of MarA (AraC) family proteins should show relatively poor inhibition of SlyA-DNA binding. In order to determine the inhibition of SlyA-DNA binding by the transcription factor modulating compounds of the invention, the MarR family member SlyA was cloned, expressed and purified from E. coli. The purified protein was used in a cell-free system to monitor DNA-protein interactions in vitro, methods as in Example 2. Compounds J and V exhibited good inhibition of SlyA-DNA binding (e.g., the IC₅₀ was greater than 25.0 μM and less than 50.0 μM).

### Example 15

### Acute P. aeruginosa Pneumonia Models

Approximately 30 Swiss Webster mice (females, 18-24 grams) are randomized to one of 4 groups of 5-10 mice per group. Animals are briefly anesthetized by isofluorane inhalation for 10-30 seconds in order to minimize the stress during intranasal inoculation. The mice are infected intranasally with 1×10⁶ P. aeruginosa bacteria diluted in room temperature sterile phosphate buffered saline (PBS) in a volume of 50 μL; a control group receives intranasal PBS with no bacteria. The mice are allowed to recover in an inclined position to improved infection efficacy. The mice are dosed IP with 25 mg/kg of the test compound in a maximum volume of 10 mL/kg (or equal volume of 5% PEG400, 95% H₂O vehicle alone) at −1, 2, 5, 20, 26, 44 and 50 hours post-infection. Infected mice are monitored for morbidity and survival twice daily over the course of 7 days. Any mice exhibiting signs of severe illness, e.g., 20% loss of their starting body weight, severe ataxia, shaking, labored breathing, unresponsiveness, etc., are painlessly euthanized by CO₂ narcoses and cervical dislocation and marked as dead. Mice infected with this inoculum of wild type P. aeruginosa (PA103) typically succumb to the infection within 48-72 hours, whereas mice infected with an ExsA null mutant strain (PA103 ΔExsA) survive indefinitely. Compounds are also tested by IV or PO administration with dose level and schedule determined from PK evaluation by these routes.

In experiments where the determination of bacterial burden in individual organs is desired, mice are infected intranasally with ˜4×10⁵ P. aeruginosa bacteria and receive the −1, 2, and 5 hour doses of compound or vehicle control. At 18 hours post infection, all mice are euthanized by CO₂ narcoses and cervical dislocation. Blood is collected immediately via cardiac puncture, and the liver, spleen and lungs are collected and weighed aseptically. Organs are homogenized in sterile PBS, and tissue and blood are plated in serial dilutions on rich media, and incubated at 37° C. for 24 hours to determine bacterial counts. In this model, infection with wild type (PA103) P. aeruginosa results in a lung bacterial burden greater than the inoculum with detectable dissemination to the peripheral tissues. Mice are not expected to develop pronounced illness in this model, but if any animals become severely moribund, they are euthanized immediately (as described previously) and marked as dead. In this model, the bacterial counts in the lungs and peripheral organs in mice infected with ExsA null mutant bacteria (PA103ΔExsA) are typically at least 2 logs lower than for mice infected with wild type (PA103) bacteria.

### EQUIVALENTS

Those skilled in the art will recognize, or be able to ascertain using no more than routine experimentation, numerous equivalents to the specific polypeptides, nucleic acids, methods, assays and reagents described herein. Such equivalents are considered to be within the scope of this invention and are covered by the following claims.

1. A transcription factor modulating compound of formula (I), formula (II), formula (III), formula (IV), formula (IVa), formula (VI), formula (VIa), formula (VII) or formula (VIII): 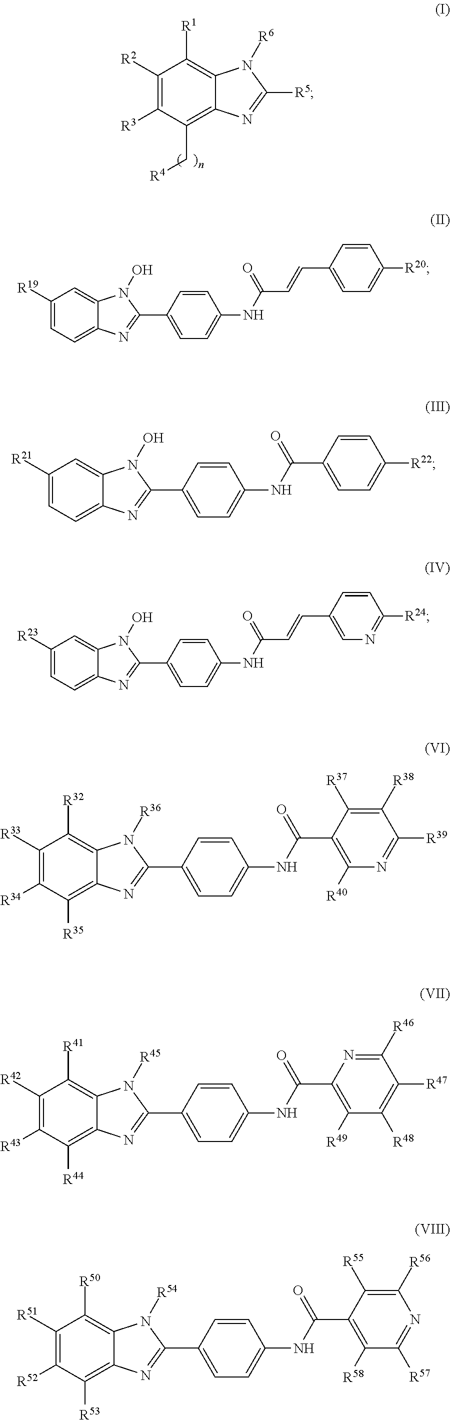
wherein
R¹, R², R³, and R⁵ are each independently hydrogen, hydroxyl, alkyl, alkenyl, alkynyl, aryl, amino, sulfonyl, carbonyl, carboxy, alkoxy, aryloxy, halogen, acyl, oximyl, hydrazinyl, —NO₂, —CN, a heterocyclic moiety or thioether;
R⁴ is —COOR⁷, —SO₃R⁸ or —PO(OR⁹)₂;
R⁶ is hydrogen, hydroxy, alkoxy, alkyl, alkenyl, alkynyl, aryl, carbonyl, carboxy or acyl;
R⁷ is hydrogen, alkyl, alkenyl, alkynyl, aryl, a heterocyclic moiety or carbonyl;
R⁸ and R⁹ are each independently hydrogen, alkyl, alkenyl, alkynyl or aryl;
n is an integer of between 1 and 10;
R¹⁹ is —COOH, —Cl, —NO₂, —CN, —SCH₃, —COCH₃, —F, —SO₂CH₃, —H or —CF₃;
R²⁰ is —COCH₃, —SCH₃ or —C(OH)₂CF₃
R²¹ is —NO₂ or —CN;
R²² is —C(OH)₂CF₃;
R²³ and R²⁴ are each independently hydrogen, hydroxyl, alkyl, alkenyl, alkynyl, aryl, amino, sulfonyl, carbonyl, carboxy, alkoxy, aryloxy, halogen, acyl, oximyl, hydrazinyl, —NO₂, —CN, a heterocyclic moiety or thioether;
R³², R³³, R³⁴, R³⁵, R³⁷, R³⁸, R³⁹ and R⁴⁰ are each independently hydrogen, hydroxyl, alkyl, alkenyl, alkynyl, aryl, amino, sulfonyl, carbonyl, carboxy, alkoxy, aryloxy, halogen, acyl, oximyl, hydrazinyl, —NO₂, —CN, a heterocyclic moiety or thioether;
R³⁶ is hydrogen, hydroxy, alkoxy, alkyl, alkenyl, alkynyl, aryl, carbonyl, carboxy or acyl;
R⁴¹, R⁴², R⁴³, R⁴⁴, R⁴⁶, R⁴⁷, R⁴⁸ and R⁴⁹ are each independently hydrogen, hydroxyl, alkyl, alkenyl, alkynyl, aryl, amino, sulfonyl, carbonyl, carboxy, alkoxy, aryloxy, halogen, acyl, oximyl, hydrazinyl, —NO₂, —CN, a heterocyclic moiety or thioether;
R⁴⁵ is hydrogen, hydroxy, alkoxy, alkyl, alkenyl, alkynyl, aryl, carbonyl, carboxy or acyl
R⁵⁰, R⁵¹, R⁵², R⁵³, R⁵⁵, R⁵⁶, R⁵⁷ and R⁵⁸ are each independently hydrogen, hydroxyl, alkyl, alkenyl, alkynyl, aryl, amino, sulfonyl, carbonyl, carboxy, alkoxy, aryloxy, halogen, acyl, oximyl, hydrazinyl, —NO₂, —CN, a heterocyclic moiety or thioether;
R⁵⁴ is hydrogen, hydroxy, alkoxy, alkyl, alkenyl, alkynyl, aryl, carbonyl, carboxy or acyl;
and pharmaceutically acceptable salts thereof.

2-53. (canceled)

54. The compound of claim 1, wherein said compound of formula (IV) is a compound of formula (IVa): 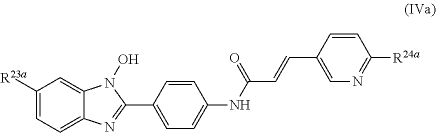
wherein
R^{23a} is —NO₂ or —CN; and
R^{24a} is acyl, thioether or amino; and pharmaceutically acceptable salts thereof.

55-74. (canceled)

75. The compound of claim 1, wherein said compound of formula (VI) is a compound of compound (VIa): 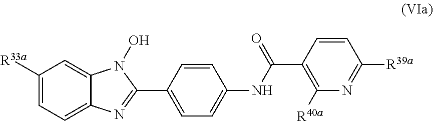
wherein
R^{33a} is —NO₂ or —CN;
R^{39a} is R^{39a} is aryl, alkyl, a heterocyclic moiety, alkyoxy or amino; and
R^{40a} is alkyl or hydrogen; and pharmaceutically acceptable salts thereof.

76-85. (canceled)

86. The compound of claim 1, wherein said compound is: 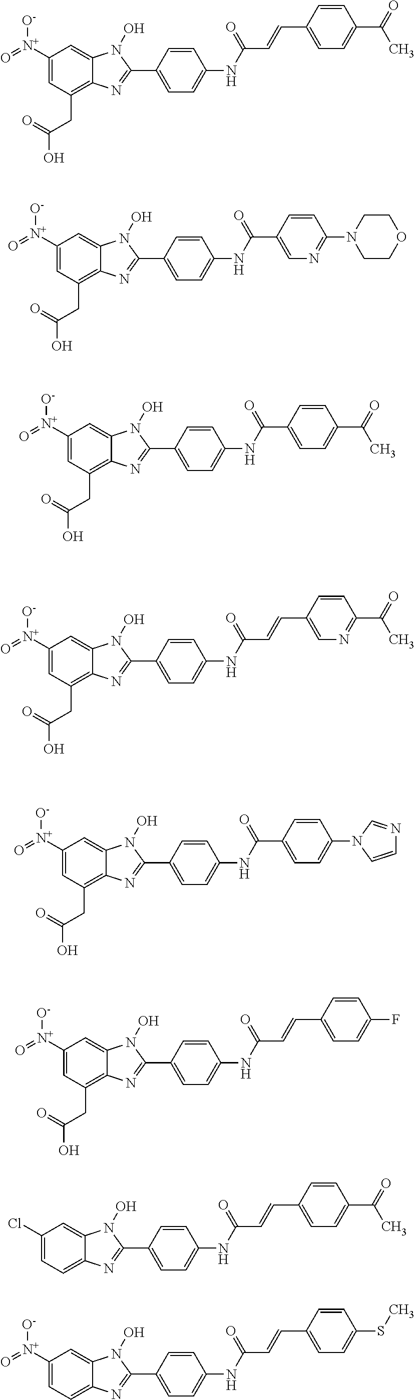 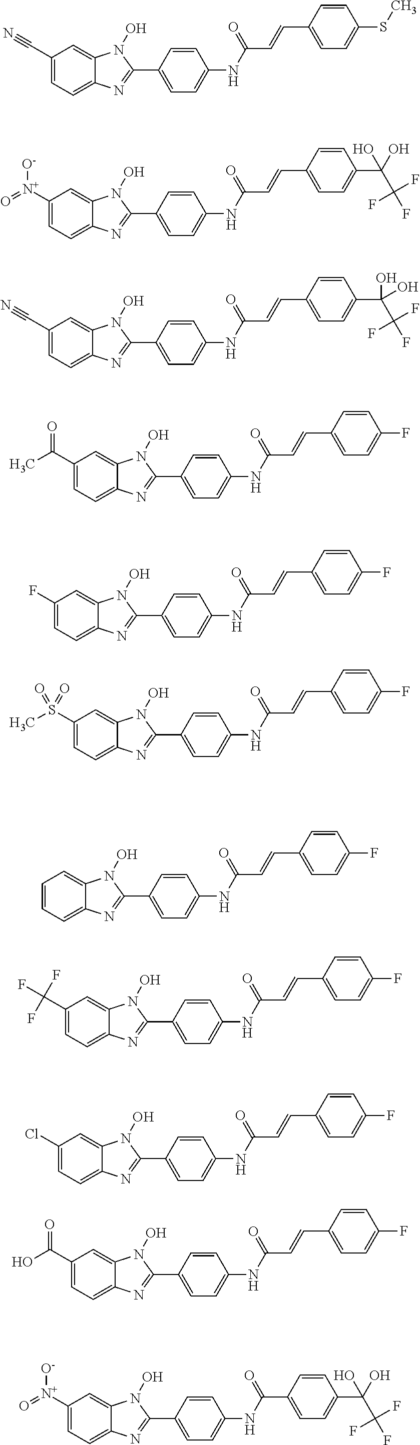 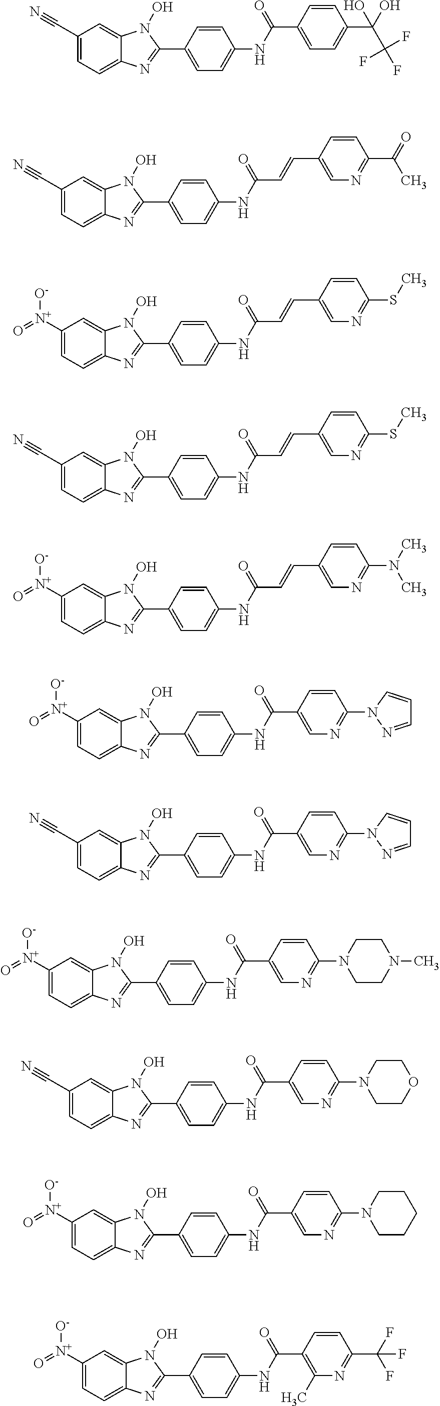 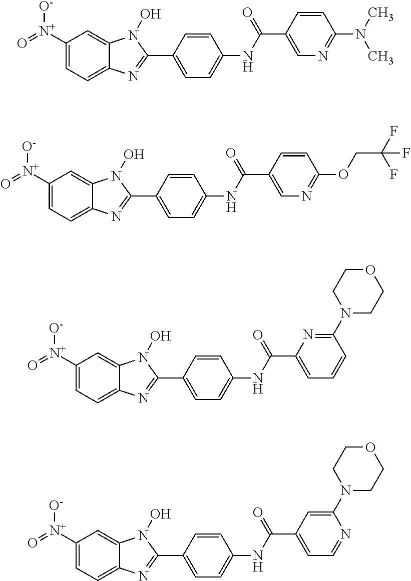
or a pharmaceutically acceptable salt thereof.

87. A pharmaceutical composition comprising a compound of claim 1 and a pharmaceutically acceptable carrier.

88. A method for reducing infectivity and/or virulence of a microbial cell, comprising contacting the cell with an effective amount of a transcription factor modulating compound of formula (I), formula (II), formula (III). formula (IV), formula (IVa), formula (VI), formula (VIa), formula (VII) or formula (VIII) such that said infectivity and/or virulence of a microbial cell is reduced.

89-95. (canceled)

96. The method of claim 88, wherein said microbial cell is P. aeruginosa, Y. pestis or Y. pseudotuberculosis.

97-102. (canceled)

103. The method of claim 88, wherein said transcription factor modulating compound is administered with a pharmaceutically acceptable carrier.

104. The method of claim 88, wherein said subject is a mammal.

105. The method of claim 104, wherein said subject is a human.

106. The method of claim 105, wherein said subject is immunocompromised.

107. The method of claim 88, wherein the transcription factor modulating compound is administered in combination with an antibiotic.
